SEQUENCE LISTING (54 sequences)

SEQ ID NO: 1 - DNA, 27 bp, Artificial Sequence
  Primer hIL-8 FW
gccaacacagaaattattgtaaagctt

SEQ ID NO: 2 - DNA, 21 bp, Artificial Sequence
  Primer hIL-8 RV
cctctgcacccagttttcctt

SEQ ID NO: 3 - DNA, 21 bp, Artificial Sequence
  Primer hTNFa FW
tggtatgagcccatctatctg

SEQ ID NO: 4 - DNA, 21 bp, Artificial Sequence
  Primer hTNFa RV
agactcggcaaagtcgagata

SEQ ID NO: 5 - DNA, 18 bp, Artificial Sequence
  Primer hIKBa FW
cgcacctccactccatcc

SEQ ID NO: 6 - DNA, 25 bp, Artificial Sequence
  Primer IKBa RV
agccatggatagaggctaagtgtag

SEQ ID NO: 7 - DNA, 19 bp, Artificial Sequence
  Primer BetaActin FW
gccaaccgcgagaagatga

SEQ ID NO: 8 - DNA, 20 bp, Artificial Sequence
  Primer BetaActin RV
ccatcacgatgccagtggta

SEQ ID NO: 9 - DNA, 19 bp, Artificial Sequence
  Primer MCP1 FW
gctcgctcagccagatgca

SEQ ID NO: 10 - DNA, 22 bp, Artificial Sequence
  Primer MCP1 RV
ggacacttactgctggtgattc

SEQ ID NO: 11 - DNA, 20 bp, Artificial Sequence
  Primer FWD MMP9
ttgacagcgacaagaagtgg

SEQ ID NO: 12 - DNA, 20 bp, Artificial Sequence
  Primer REV MMP9
gccattcacgtcgtccttat

SEQ ID NO: 13 - DNA, 24 bp, Artificial Sequence
  Primer FWD Bcl2
ttgtggccttctttgagttcggtg

SEQ ID NO: 14 - DNA, 24 bp, Artificial Sequence
  Primer Rev Bcl2
gtacagttccacaaaggcatccca

SEQ ID NO: 15 - DNA, 21 bp, Artificial Sequence
  Primer IL-6F RT
ggtacatcctcgacggcatct

SEQ ID NO: 16 - DNA, 21 bp, Artificial Sequence
  Primer IL-6R RT
gtgcctctttgctgctttcac

SEQ ID NO: 17 - DNA, 20 bp, Artificial Sequence
  Primer Fwd TERT
gactacgtcgtgggagccag

SEQ ID NO: 18 - DNA, 21 bp, Artificial Sequence
  Primer Rev TERT
cctgtggatatcgtccaggcc

SEQ ID NO: 19 - DNA, 24 bp, Artificial Sequence
  Primer hTERC FWD
tctaaccctaactgagaagggcgt

SEQ ID NO: 20 - DNA, 24 bp, Artificial Sequence
  Primer hTERC REV
tgctctagaatgaacggtggaagg

SEQ ID NO: 21 - DNA, 20 bp, Artificial Sequence
  Primer STAT3 qPCR FWD
gatccagtccgtggaaccat

SEQ ID NO: 22 - DNA, 23 bp, Artificial Sequence
  Primer STAT3 qPCR REV
atagcccatgatgatttcagcaa

SEQ ID NO: 23 - DNA, 20 bp, Artificial Sequence
  Primer mBetaActin FW
ctgacggccaggtcatcact

SEQ ID NO: 24 - DNA, 23 bp, Artificial Sequence
  Primer mBetaActin RV
tagtttcatggatgccacaggat

SEQ ID NO: 25 - DNA, 18 bp, Artificial Sequence
  Primer mIKBa FW
ggccagctgaccctggaa

SEQ ID NO: 26 - DNA, 21 bp, Artificial Sequence
  Primer mIKBa RV
gcctccaaacacacagtcatc

SEQ ID NO: 27 - DNA, 22 bp, Artificial Sequence
  Primer mIL6 FW
caaagccagagtccttcagaga

SEQ ID NO: 28 - DNA, 21 bp, Artificial Sequence
  Primer mIL6 RV
gccactccttctgtgactcca

SEQ ID NO: 29 - DNA, 22 bp, Artificial Sequence
  Primer mMCP1 FW
gcccctccatgtataccagact

SEQ ID NO: 30 - DNA, 22 bp, Artificial Sequence
  Primer mMCP1 RV
agacctctctcttgagcttggt

SEQ ID NO: 31 - DNA, 20 bp, Artificial Sequence
  Primer mIL6 FW2
gttgtgcaatggcaattctg

SEQ ID NO: 32 - DNA, 21 bp, Artificial Sequence
  Primer mIL6 RV2
ctctgaaggactctggctttg

SEQ ID NO: 33 - DNA, 20 bp, Artificial Sequence
  Primer mIL1 FW
aaagctctccacctcaatgg

SEQ ID NO: 34 - DNA, 21 bp, Artificial Sequence
  Primer mIL1 RV
tcttctttgggtattgcttgg

SEQ ID NO: 35 - DNA, 21 bp, Artificial Sequence
  Primer mTNF FW2
acagaaagcatgatccgcgac

SEQ ID NO: 36 - DNA, 19 bp, Artificial Sequence
  Primer mTNF RV2
gaagccccccatcttttgg

SEQ ID NO: 37 - DNA, 21 bp, Artificial Sequence
  Primer mTERC FWD
tttgttctccgcccgctgttt

SEQ ID NO: 38 - DNA, 23 bp, Artificial Sequence
  Primer mTERC REV
agctcctgcgctgacgtttgttt

SEQ ID NO: 39 - DNA, 27 bp, Artificial Sequence
  NFkB consensus probe
tcaacagaggggactttccgagaggcc

SEQ ID NO: 40 - DNA, 33 bp, Artificial Sequence
  IL6A probe
actgggaggattcccaaggggtcaattgggaga

SEQ ID NO: 41 - DNA, 25 bp, Artificial Sequence
  IL6B  probe
actgggaggattcccaaggggtcaa

SEQ ID NO: 42 - DNA, 30 bp, Artificial Sequence
  IL8 A  probe
tgactcaggtttgccctgaggggatgggcc

SEQ ID NO: 43 - DNA, 30 bp, Artificial Sequence
  IL8 B  probe
tgactcaggcccgccctgaggggatgggcc

SEQ ID NO: 44 - DNA, 44 bp, Artificial Sequence
  TNF-alpha A  probe
gcatgggaatttccaactctgggaattccaatccttgctgggaa

SEQ ID NO: 45 - DNA, 44 bp, Artificial Sequence
  TNF-alpha B probe
gcatgggaatttccaactctcccaaccccaatccttgctgggaa

SEQ ID NO: 46 - DNA, 22 bp, Artificial Sequence
  Oct-1 probe
tgtcgaatgcaaatcactagaa

SEQ ID NO: 47 - protein, 26 aa, Artificial Sequence
  NFkB inhibitor sc-3060
AAVALLPAVLLALLAPVQRKRQKLMP

SEQ ID NO: 48 - DNA, 21 bp, Artificial Sequence
  Telomerase inhibitor Sh-hTERT
catttcatcagcaagtttgga

SEQ ID NO: 49 - DNA, 19 bp, Artificial Sequence
  Telomerase inhibitor Sh-hTerc
gtctaaccctaactgagaa

SEQ ID NO: 50 - RNA, 19 nt, Artificial Sequence
  telomerase inhibitor siTERT 1
gaacgggccuggaaccaua

SEQ ID NO: 51 - RNA, 19 nt, Artificial Sequence
  Telomerase inhibitor siTERT 2
cgccugagcuguacuuugu

SEQ ID NO: 52 - RNA, 19 nt, Artificial Sequence
  telomerase inhibitor siTERT 3
gguaugccgugguccagaa

SEQ ID NO: 53 - RNA, 19 nt, Artificial Sequence
  telomerase inhibitor siTERT 4
gcgacgacgugcugguuca

SEQ ID NO: 54 - DNA, 19 bp, Artificial Sequence
  NFkB inhibitor shReIA
agccattagccagcgaatc

(19) United States
(12) Patent Application Publication
(10) Pub. No.: US 2015/0232856 A1
(43) Pub. Date: Aug. 20, 2015

(54) TELOMERASE INHIBITORS FOR USE IN THERAPY

(71) Applicant: AGENCY FOR SCIENCE, TECHNOLOGY AND RESEARCH, Singapore (SG)

(72) Inventor: Vinay Tergaonkar, Proteos (SG)

(21) Appl. No.: 14/431,215

(22) PCT Filed: Sep. 25, 2013
(86) PCT No.: PCT/SG2013/000418

§ 371 Date: Mar. 25, 2015

(30) Foreign Application Priority Data
Sep. 25, 2012 (SG) 201207102-3

(51) Int. Cl. C12N 15/113; A61K 31/7105; A61K 31/713; A61K 39/395; A61K 31/167; C07K 16/40

(52) U.S. Cl. CPC C12N 15/1137; A61K 31/167; C07K 16/40; A61K 31/713; A61K 39/3955; A61K 31/7105; C12N 2310/14; C12N 2320/30; C07K 2317/76

(57) ABSTRACT

The present invention relates to methods of treating an inflammatory disease and/or cancer in a patient in need thereof, the method comprising administering a telomerase inhibitor to the patient.

### TECHNICAL FIELD

The present invention generally relates to methods of treating inflammatory diseases and cancer.

### BACKGROUND

Telomeres are tandem repeats of (TTAGGG)n sequence at the ends of chromosomes bound by a complex of proteins known as the “shelterin complex”. This complex is thought to protect telomeres from degradation and DNA repair activities^{1,2}. Telomere length is maintained and replenished by the ribonucleoprotein enzyme “telomerase”^{3,4}. Growing evidence suggests that mature telomerase⁵⁻¹⁰, as well as shelterin complex members¹¹⁻¹⁴ are all also involved in non-canonical activities at extra-telomeric sites or organelles^{5,13,15-19}.

Elongation of chromosomal ends by telomerase²⁰ prevents senescence and allows cells to overcome the Hayflick limit²¹. Telomerase is reactivated in 80-90% of all cancers,²² and in some other human diseases apart from cancers^{5,23,24}. Although it is assumed that elongation of telomeres is the primary function of reactivated telomerase in human cancers²⁵⁻²⁷, this activity of telomerase does not account for all the properties such as increased cell proliferation, increased resistance to apoptosis and increased invasion seen in human cancer cells. The mechanistic basis and reason for telomerase reactivation, as well as the molecular mechanisms (if any) which link the non-canonical activities of telomerase to the acquired phenotypes of cancer cells are still not understood.

Weinberg and colleagues observed that ALT (alternate lengthening of telomeres) mediated telomere maintenance failed to substitute for telomerase in transformation and tumorigenesis, thereby initiating the idea that merely elongating telomeres is not the sole function of telomerase²⁸. Various studies since have postulated-novel roles for telomerase, which are independent of its function on the telomeres^{15,29}. Interestingly, a number of these roles are now being suggested to be important for the molecular function of reactivated telomerase in human cancers as well as in diseases like atherosclerosis⁵ and kidney dysfunction²⁴. These alternate functions include the role of telomerase in transcription, namely that of Wnt target genes¹⁰, in regulation of mitochondrial function^{8,17,30} and in cellular response to DNA damage^{9,31-33}.

Some of these novel, “non-canonical” functions of telomerase were initially described in murine studies^{34,35}. Although it was shown that mTERT (catalytic component of murine telomerase) overexpression in mice led to spontaneous tumorigenesis, it was noted that under such conditions telomere length did not change appreciably^{36,37}. Conversely, lack of telomerase led to repression of spontaneous tumorigenesis³⁸. However, murine telomeres are very long and pathologies related to telomere shortening and accelerated aging are only observed in fifth or sixth generation telomerase knockout mice³⁹⁻⁴². Although there is low-level telomerase activity in many somatic murine tissues, mTERT overexpression in breast epithelia could induce cancer^{35,43}. Furthermore, recent studies in primary human mammary epithelial cells have identified an apparently telomere-independent function of hTERT (catalytic component of human telomerase) that enables human mammary epithelial cells (HMECs) to proliferate in mitogen deficient conditions, a hallmark of cancer^{8,44}. Another study demonstrated that overexpressing mTERT in skin epithelia causes proliferation of hair follicle stem cells⁴⁵. Transgenic mice overexpressing mTERT not only show enhanced incidence of carcinogen induced tumor formation but also exhibit increased wound healing ability³⁴. Ectopic telomerase expression can protect cells from antiproliferative or apoptotic stimuli^{46,47}. Conversely, in a wide variety of cell types, telomerase inhibition can enhance sensitivity to cytotoxic drugs^{22,48,49}. Additionally, hTERT can function as a RNA dependent RNA polymerase that can bind to non-hTerc RNAs and mediate independent functions, especially in the mitochondria^{6-8,17,50}. These multitudes of alternative telomerase functions suggest that it is not just a telomere elongating enzyme.

Another hallmark of most human cancers is inflammation. A key driver of inflammation is NFκB signalling. NFκB is a transcription factor that is central to the function of several cellular and developmental signalling pathways⁵¹⁻⁵⁶. NFκB is a “master” regulator of genes involved in proliferation, resistance to apoptosis and invasion. NFκB target genes include cell cycle genes like cyclin D1; inflammatory cytokines like IL6, TNF, IL8; survival genes, such as IAPs (inhibitor of apoptosis), Bcl2, A20⁵⁶; and invasion associated genes such as MMP9 and ICAM1⁵⁷. NFκB activation is associated with tumorigenesis^{18,58} and with high Ki67 index and tumor grades in cancers^{13,59}. Activated nuclear NFκB is a hallmark of tumors' resistance to anti-cancer drugs⁶⁰⁻⁶². Under resting state, IκB proteins inhibit NFκB function by preventing NFκB DNA binding. Stimulus dependent phosphorylation of IκBα is mediated by the IκB kinases (IKK1 and IKK2) which reside in a complex along with chaperones such as ELKS and NEMO (IKKγ)⁶³. Phosphorylated IκB proteins are ubiquitinated and degraded^{52,57} largely in the cytoplasm⁶⁴. NFκB subunits free from IκB proteins accumulate in the nucleus and bind DNA at NFκB sites⁶⁵. Uncontrolled activation of NFκB and its effects such as chronic inflammation are hallmarks of many human cancers but the mechanism of how NFκB activity is sustained in cancers is unknown. Activation of additional co-signalling modules involved in carcinogenesis which feed forward NFκB dependent signalling could be one possible explanation.

There is a need to provide methods for inhibiting NFκB activation to thereby prevent and treat inflammation and progression to cancer, and its recurrence.

### SUMMARY

The present invention is based on the surprising finding that telomerase inhibitors inhibit regulatory elements of NFκB mediated inflammation. The ability of telomerase inhibitors to block inflammation enables their use in the treatment of inflammatory diseases as well as cancer, which is often characterized by inflammation.

In a first aspect, there is provided a method of treating an inflammatory disease and/or cancer in a patient in need thereof, the method comprising administering a telomerase inhibitor to the patient.

Advantageously, telomerase only affects about 13% of NFκB target genes, and hence the use of telomerase inhibitors confers specificity towards these target genes. It was also found that telomerase strongly inhibits IL6 and TNF which are key cytokines involved in human diseases ranging from inflammation to cancer and metabolic syndromes. In addition, blocking telomerase which is expressed to undetectable levels in most human cells except stem cells of highly proliferating organs, avoids off target effects. Toxicity seen with anti-inflammatory drugs (e.g. NSAIDs) can also be avoided with use of telomerase inhibitors.

In a second aspect, there is provided a method of sensitizing a patient to treatment with an anti-inflammatory drug and/or an anti-cancer drug, the method comprising administering a telomerase inhibitor to the patient.

In a third aspect, there is provided a method of preventing recurrence of an inflammatory disease and/or cancer in a patient in need thereof, the method comprising administering a telomerase inhibitor to the patient.

In a fourth aspect, there is provided a pharmaceutical composition comprising a telomerase inhibitor and a pharmaceutically acceptable excipient, for use in treating an inflammatory disease and/or cancer.

In a fifth aspect, there is provided a telomerase inhibitor for use in treating or preventing recurrence of an inflammatory disease and/or cancer in a patient in need thereof, or for sensitizing a patient to treatment with an anti-inflammatory drug and/or an anti-cancer drug.

In a sixth aspect, there is provided a use of a telomerase inhibitor in the manufacture of a medicament for treating or preventing recurrence of an inflammatory disease and/or cancer in a patient in need thereof, or for sensitizing a patient to treatment with an anti-inflammatory drug and/or an anti-cancer drug.

### DEFINITIONS

The following words and terms used herein shall have the meaning indicated:

The term “nucleic acid” is to be interpreted broadly to include a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. The terms “nucleic acid”, “nucleic acid agent”, “nucleic acid molecule”, “nucleic acid sequence” and polynucleotide etc. are used interchangeably herein unless the context indicates otherwise.

The term “treatment” includes any and all uses which remedy a disease state or symptoms, prevent the establishment of disease, or otherwise prevent, hinder, retard, or reverse the progression of disease or other undesirable symptoms in any way whatsoever. Hence, “treatment” includes prophylactic and therapeutic treatment. Beneficial or desired clinical results include, but are not limited to, alleviation of symptoms; diminishment of extent of condition, disorder or disease; stabilized (i.e. not worsening) state of condition, disorder or disease; delay or slowing of condition, disorder or disease progression; amelioration of the condition, disorder or disease state; remission (whether partial or total, and whether detectable or undetectable); or enhancement or improvement of condition, disorder or disease. Treatment includes eliciting a cellular response that is clinically significant, without excessive levels of side effects. Treatment also includes prolonging survival as compared to expected survival if not receiving treatment. Treatment may entail treatment with a single agent or with a combination (two or more) of agents. An “agent” is used herein broadly to refer to, for example, a compound or other means for treatment e.g. radiation treatment or surgery.

The term “sensitize” as used herein, for therapeutic purposes, generally refers to causing a patient to be susceptible to treatment with a single agent or with a combination (two or more) of agents to thereby allow for more effective treatment of a disease. For example, sensitizing a patient to treatment with an anti-cancer drug refers to causing the patient to be susceptible to treatment with the anti-cancer drug, and sensitizing a patient to treatment with an anti-inflammatory drug refers to causing the patient to be susceptible to treatment with the anti-inflammatory drug.

As used herein, the term “therapeutically effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

In the context of this invention the term “administering” and variations of that term including “administer” and “administration”, includes contacting, applying, delivering or providing a compound or composition of the invention to an organism, or a surface by any appropriate means.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DISCLOSURE OF OPTIONAL EMBODIMENTS

Exemplary, non-limiting embodiments of a method of treating inflammatory diseases or cancer, will now be disclosed.

The present invention is based on the surprising finding that telomerase inhibitors inhibit regulatory elements of NFκB inflammation. This allows the use of telomerase inhibitors to block inflammation, and thereby treat inflammatory disease. Furthermore, the ability of telomerase inhibitors to block inflammation also allows their use in the treatment of cancer.

The inventors have found a novel link between telomerase and NFκB, and a novel role for telomerase in direct regulation of NFκB dependent genes in cancer cells. These findings may explain how telomerase reactivation may be critical for cancer progression due, in part, to its ability to feed forward a constitutive NFκB dependent gene expression program in cancer tissues.

It was observed that while blocking NFκB signalling can inhibit effects of telomerase overexpression on processes relevant to transformation, increasing NFκB activity can functionally substitute for reduced telomerase activity and its effects on these cellular processes. The inventors found that telomerase directly regulates NFκB dependent gene expression, and that mice lacking either Terc (telomerase RNA component) or TERT components and hence functional telomerase activity have dampened NFκB signalling and inflammatory responses. Biochemical and genome wide. CHIP-seq data revealed that the ability of telomerase to activate NFκB dependent gene expression is a function of its binding to the NFκB p65 subunit and recruitment to a subset of NFκB promoters such as that of IL6 which is a key cytokine critical for inflammation and cancer progression. Interestingly, there is a significant overlap in the number of cancers which show both telomerase reactivation and constitutive activation of NFκB. Given that NFκB has been previously documented to upregulate telomerase levels, the inventors' findings provide a molecular explanation for co-regulation of these pathways in cancers, and suggest that the feed-forward regulation between these two pathways could provide a key mechanistic basis for co-existence of chronic inflammation and sustained telomerase activity in human cancers.

In a first aspect, there is provided a method of treating an inflammatory disease and/or cancer in a patient in need thereof, the method comprising administering a telomerase inhibitor to the patient.

The treatment may include: (i) the prevention or inhibition of recurrence of the inflammatory disease and/or cancer, (ii) the reduction or elimination of symptoms or cancer cells, and (iii) the substantial or complete elimination of the inflammatory disease and/or cancer in question. Treatment may be effected prophylactically (prior to disease onset) or therapeutically (following disease diagnosis).

Hence, in one aspect, there is also provided a method of preventing recurrence of an inflammatory disease and/or cancer in a patient in need thereof, the method comprising administering a telomerase inhibitor to the patient.

In another aspect, there is provided a method of sensitizing a patient to treatment with an anti-inflammatory drug and/or an anti-cancer drug, the method comprising administering a telomerase inhibitor to the patient.

An “inhibitor” as used herein includes any molecule which decreases the activity of the target molecule, for example by interfering with interaction of the target molecule with another molecule (e.g., its substrate), or decreases the protein level of the target molecule, for example by decreasing expression of the gene encoding the target molecule. An inhibitor may be a “direct inhibitor” which interacts with the target molecule or binding partner thereof or with a nucleic acid encoding the target molecule, or an “indirect inhibitor” which does not interact with the target molecule or binding partner thereof or with a nucleic acid encoding the target molecule, but rather interacts upstream or downstream of the target molecule in the regulatory pathway.

Hence, a “telomerase inhibitor” includes any molecule which decreases the activity of telomerase or decreases the protein level of telomerase. Thus, a telomerase inhibitor can be a molecule which decreases activity of telomerase, for example by interfering with interaction of telomerase with another molecule, e.g., its substrate. It can also be a molecule which decreases expression of the gene encoding telomerase. The telomerase inhibitor may be a “direct inhibitor” or an “indirect inhibitor” as defined above.

The telomerase inhibitor can be selected from the group consisting of interfering nucleic acid agent, an antibody, a small inorganic molecule, and peptide nucleic acids (PNA).

An interfering nucleic acid agent as used herein can be a double stranded RNA (dsRNA) or an antisense. RNA or a ribozyme.

The dsRNA can include, but is not limited to, short hairpin RNA (shRNA), small interfering (siRNA), and micro RNA (miRNA). siRNA can be between about 15-30 nucleotides in length. siRNA can comprise 1-3 nucleotide overhangs at the 3′ and 5′ termini. The dsRNA and/or antisense RNA can be modified to comprise modified nucleotides selected from the group consisting of 2′-O-methyl (2′OMe) nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof. For example, the modified nucleotides can comprise 2′OMe nucleotides selected from the group consisting of 2′OMe-guanosine nucleotides, 2′OMe-uridine nucleotides, 2′OMe-adenosine nucleotides, and mixtures thereof.

In one embodiment, the telomerase inhibitor is a 2′-O-alkyl oligonucleotide inhibitor.

In one embodiment, the telomerase inhibitor is a dsRNA, such as shRNA directed against hTERT (such as sh-TERT).

In one embodiment, the sh-TERT is a sh-hTERT comprising SEQ ID NO: 48 (CATTTCATCAGCAAGTTTGGA). In one embodiment, the sh-TERT is a sh-hTERT consisting of SEQ ID NO: 48 (CATTTCATCAGCAAGTTTGGA).

In one embodiment, the telomerase inhibitor is a dsRNA, such as shRNA directed against hTerc (such as sh-Terc).

In one embodiment, the sh-Terc is a sh-hTerc comprising SEQ ID NO: 49 (GTCTAACCCTAACTGAGAA). In one embodiment, the sh-Terc is a sh-hTerc consisting of SEQ ID NO: 49 (GTCTAACCCTAACTGAGAA). In one embodiment, the telomerase inhibitor is a dsRNA, such as siRNA directed against hTERT (such as si-hTERT).

In one embodiment, the si-hTERT comprises a sequence selected from, the group consisting of SEQ ID NO: 50 (GAACGGGCCUGGAACCAUA), SEQ ID NO: 51 (CGCCUGAGCUGUACUUUGU), SEQ ID NO: 52 (GGUAUGCCGUGGUCCAGAA) and SEQ ID NO: 53 (GCGACGACGUGCUGGUUCA). In one embodiment, the si-hTERT consists of a sequence selected from the group consisting of SEQ ID NO: 50 (GAACGGGCCUGGAACCAUA), SEQ ID NO: 51 (CGCCUGAGCUGUACUUUGU), SEQ ID NO: 52 (GGUAUGCCGUGGUCCAGAA) and SEQ ID NO: 53 (GCGACGACGUGCUGGUUCA).

In one embodiment, the telomerase inhibitor is a dsRNA, such as siRNA directed against hTerc (such as si-hTerc). Suitable interfering nucleic acid agents as used herein can be manufactured by chemical synthesis, recombinant DNA procedures or, in the case of antisense RNA, by transcription in vitro or in vivo when linked to a promoter, by methods known to those skilled in the art. For example, siRNA is typically generated by cleavage of double stranded RNA, where one strand is identical to the message to be inactivated. Double-stranded RNA molecules may be synthesised in which one strand is identical to a specific region of the mRNA transcript and introduced directly. Alternatively, corresponding dsDNA can be employed, which, once presented intracellularly is converted into dsRNA. Methods for the synthesis of suitable siRNA molecules for use in RNA interference and for achieving post-transcriptional gene silencing are known to those of skill in the art. The skilled addressee will appreciate that a range of suitable siRNA constructs capable of inhibiting the expression of the gene(s) encoding the target molecule can be identified and generated based on knowledge of the sequence of the gene(s) in question using routine procedures known to those skilled in the art without undue experimentation. In one example, the si-hTerc can be a commercially available siRNA, for example from Dharmacon.

Those skilled in the art will also appreciate that there need not necessarily be 100% nucleotide sequence match between the target sequence and the siRNA sequence. The capacity for mismatch is dependent largely on the location of the mismatch within the sequences. In some instances, mismatches of 2 or 3 nucleotides may be acceptable but in other instances a single nucleotide mismatch is enough to negate the effectiveness of the siRNA. The suitability of a particular siRNA molecule may be determined using routine procedures known to those skilled in the art without undue experimentation.

In one embodiment, the telomerase inhibitor is an interfering nucleic agent such as an antisense RNA. Sequences of antisense constructs may be derived from various regions of the telomerase gene.

Antisense constructs as used herein may be designed to target and bind to regulatory regions of the nucleotide sequence, such as the promoter, or to coding (exon) or non-coding (intron) sequences. For example, to reduce expression of the telomerase gene, antisense oligonucleotides may be designed to target hTERT or hTerc, and may be designed to be complementary for any suitable portion of these components.

Antisense constructs of the invention may be generated which are at least substantially complementary across their length to the region of the gene in question. Binding of an antisense construct to its complementary, cellular sequence may interfere with transcription, RNA processing, transport, translation and/or mRNA stability.

Suitable antisense oligonucleotides may be prepared by methods well known to those of skill in the art. Typically antisense oligonucleotides will be synthesized on automated synthesizers. Suitable antisense oligonucleotides may include modifications designed to improve their delivery into cells, their stability once inside a cell, and/or their binding to the appropriate target. For example, the antisense oligonucleotide may be modified by the addition of one or more phosphorothioate linkages, or the inclusion of one or morpholine rings into the backbone.

A further means of substantially, inhibiting gene expression may be achieved by introducing catalytic antisense nucleic acid constructs, such as ribozymes, which are capable of cleaving RNA transcripts and thereby preventing the production of wildtype protein. Ribozymes are targeted to and anneal with a particular sequence by virtue of two regions of sequence complementarity to the target flanking the ribozyme catalytic site. After binding, the ribozyme cleaves the target in a site-specific manner. The design and testing of ribozymes which specifically recognize and cleave sequences of interest can be achieved by techniques well known to those in the art (for example Lieber and Strauss, (1995) Mol. Cell. Biol. 15:540-551, the disclosure of which is incorporated herein by reference).

An interfering nucleic acid agent of the invention may be administered in a vector. The vector may be a plasmid vector, a viral vector, or any other suitable vehicle adapted for the insertion of foreign sequences and introduction into eukaryotic cells. In one example, the vector is an expression vector capable of directing the transcription of the DNA sequence of an interfering nucleic acid agent into RNA. Viral expression vectors include, for example, epstein-barr virus-, bovine papilloma virus-, adenovirus- and adeno-associated virus-based vectors. In one example, the vector is episomal. The use of a suitable episomal vector provides a means of maintaining the interfering nucleic acid agent in target cells in high copy number extra-chromosomally thereby eliminating potential effects of chromosomal integration.

In one embodiment, the telomerase inhibitor is an antibody capable of binding to a specific epitope on a telomerase.

In one embodiment, the telomerase inhibitor is an antibody directed against hTERT (such as anti-hTERT).

Antibodies that may be used in the present invention can comprise a polyclonal mixture, or may be monoclonal in nature. Further, the antibodies can be entire immunoglobulins derived from natural sources, or from recombinant sources. The antibodies may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, Fab, F(ab)₂, scFv (single chain Fv), dAb (single domain antibody), bi-specific antibodies, diabodies and triabodies.

In another embodiment, the telomerase inhibitor is a small molecule. A “small molecule” is an organic (having at least one carbon atom) or inorganic (having no carbon atoms) compound that has a molecular weight that is sufficiently low to allow the small molecule to rapidly diffuse across cell membranes so that they can reach intracellular sites of action. Typically, the molecular weight of a small molecule is less than about 800 g/mol (e.g. less than about 700 g/mol, less than about 600 g/mol, less than about 500 g/mol, less than about 400 g/mol, less than about 300 g/mol, less than about 200 g/mol, less than about 100 g/mol, between about 50 to about 800 g/mol, between about 100 to about 800 g/mol, between about 500 to about 800 g/mol, between about 100 to about 300 g/mol, or between about 100 to about 500 g/mol).

In one embodiment, the telomerase inhibitor is a small inorganic molecule. In some embodiments, the small inorganic molecule is a therapeutically active agent such as a drug (e.g. a small inorganic molecule approved by the U.S. Food and Drug Administration as provided in the Code of Federal Regulations (CFR)).

In one embodiment, the small inorganic molecule can be selected from the group consisting of N,N′-1,3-Phenylenebis-[2,3-dihydroxy-benzamide] (MST-312), BIBR 1532, (2-[(E)-3-naphtalen-2-yl-but-2-enoylamino]-benzoic acid), and costunolide ((3aS,6E,10E,11aR)-6,10-dimethyl-3-methylene-3,3a,4,5,8,9-hexahydrocyclodeca[b]furan-2(11aH)-one).

In yet another embodiment, the telomerase inhibitor is a peptide nucleic acid (PNA). A PNA is an antisense molecule or anti-gene agent which comprises an oligonucleotide of at least about 5 nucleotides in length linked to a peptide backbone of amino acid residues, which preferably ends in lysine. The terminal lysine confers solubility to the composition. PNAs preferentially bind complementary single stranded DNA or RNA and stop transcript elongation, and may be pegylated to extend their lifespan in the cell.

In one example, the telomerase inhibitor can be based on anyone of the following molecules:

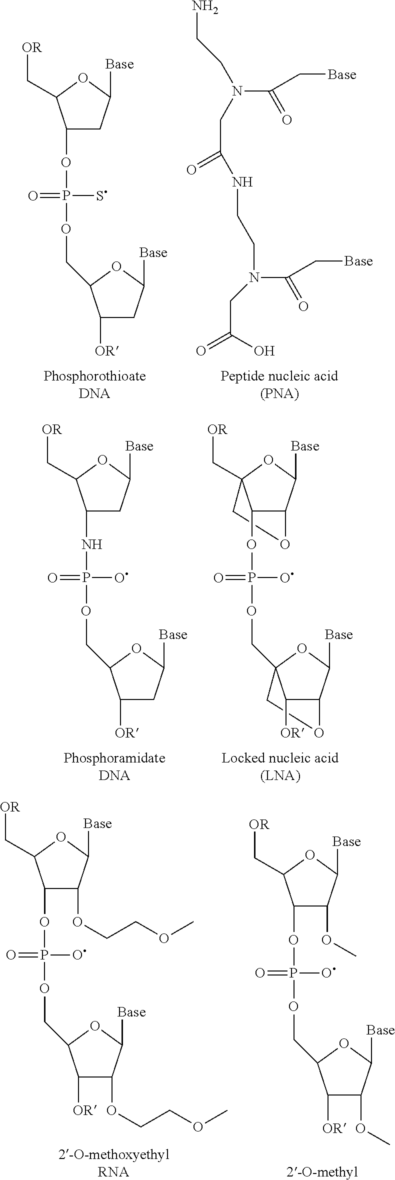

In accordance with the present invention, when used for the treatment or prevention of inflammatory disease and/or cancer, the telomerase inhibitors of the invention may be administered alone. Alternatively, the telomerase inhibitors may be administered as a pharmaceutical or veterinarial formulation which comprises at least one telomerase inhibitor according to the invention.

Hence, in one aspect, there is provided use of a telomerase inhibitor in the manufacture of a medicament for treating or preventing recurrence of an inflammatory disease and/or cancer in a patient in need thereof, or for sensitizing a patient to treatment with an anti-inflammatory drug and/or an anti-cancer drug.

In another one aspect, there is provided a pharmaceutical composition comprising a telomerase inhibitor and a pharmaceutically acceptable excipient.

In accordance with the present invention, the telomerase inhibitor(s) of the invention may be used in combination with other pharmaceutically active ingredients, or known treatments, or anti-inflammatory agents, or anti-cancer agents. Suitable agents are listed, for example, in the Merck Index, An Encyclopoedia of Chemicals, Drugs and Biologicals, 12th Ed., 1996, the entire contents of which are incorporated herein by reference.

Exemplary anti-cancer drugs include, but are not limited to, acivicin, aclarubicin, acodazole, acronycine, adozelesin, alanosine, aldesleukin, allopurinol sodium, altretamine, aminoglutethimide, amonafide, ampligen, amsacrine, androgens, anguidine, aphidicolin glycinate, asaley, asparaginase, 5-azacitidine, azathioprine, Bacillus calmette-guerin (BCG), Baker's Antifol (soluble), beta-2′-deoxythioguanosine, bisantrene hydrochloride, bleomycin sulphate, busulphan, buthionine sulphoximine, ceracemide, carbetimer, carboplatin, carmustine, chlorambucil, chloroquinoxaline-sulphonamide, chlorozotocin, chromomycin A3, cisplatin, cladribine, corticosteroids, Corynebacterium parvum, CPT-11, crisnatol, cyclocytidine, cyclophosphamide, cytarabine, cytembena, dabis maleate, dacarbazine, dactinomycin, daunorubicin HCl, deazauridine, dexrazoxane, dianhydrogalactitol, diaziquone, dibromodulcitol, didemnin B, diethyldithiocarbamate, diglycoaldehyde, dihydro-5-azacytidine, doxorubicin, echinomycin, edatrexate, edelfosine, eflomithine, Elliott's solution, elsamitrucin, epirubicin, esorubicin, estramustine phosphate, estrogens, etanidazole, ethiofos, etoposide, fadrazole, fazarabine, fenretinide, filgrastim, finasteride, flavone acetic acid, floxuridine, fludarabine phosphate, 5-fluorouracil, Fluosol, flutamide, gallium nitrate, gemcitabine, goserelin acetate, hepsulfam, hexamethylene bisacetamide, homoharringtonine, hydrazine sulphate, 4-hydroxyandrostenedione, hydrozyurea, idarubicin hydrochoride, ifosfamide, interferon alfa, interferon beta, interferon gamma, interleukin-1 alpha and beta, interleukin-3, interleukin-4, interleukin-6, 4-ipomeanol, iproplatin, isotretinoin, leucovorin calcium, leuprolide acetate, levamisole, liposomal daunorubicin, liposome encapsulated doxorubicin, lomustine, lonidamine, maytansine, mechlorethamine hydrochloride, melphalan, menogaril, merbarone, 6-mercaptopurine, mesna, methanol extraction residue of Bacillus calmette-guerin, methotrexate, N-methylformamide, mifepristone, mitoguazone, mitomycin-C, mitotane, mitoxantrone hydrochloride, monocyte/macrophage colony-stimulating factor, nabilone, nafoxidine, neocarzinostatin, octreotide acetate, ormaplatin, oxaliplatin, paclitaxel, pala, pentostatin, piperazinedione, pipobroman, pirarubicin, piritrexim, piroxantrone hydrochloride, PIXY-321, plicamycin, porfimer sodium, prednimustine, procarbazine, progestins, pyrazofurin, razoxane, sargramostim, semustine, spirogermanium, spiromustine, streptonigrin, streptozocin, sulofenur, suramin sodium, tamoxifen, taxotere, tegafur, teniposide, terephthalamidine, teroxirone, thioguanine, thiotepa, thymidine injection, tiazofurin, topotecan, toremifene, tretinoin, trifluoperazine hydrochloride, trifluridine, trimetrexate, tumor necrosis factor, uracil mustard, vinblastine sulphate, vincristine sulphate, vindesine, vinorelbine, vinzolidine, Yoshi 864, zorubicin, and combinations thereof.

Exemplary anti-inflammatory drugs include, but are not limited to, classic non-steroidal anti-inflammatory drugs (NSAIDS), such as aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone (relafen), acetaminophen (sold under the trade mark Tylenol), and mixtures thereof; COX-2 inhibitors, such as nimesulide, NS-398, flosulid, L-745337, celecoxib, rofecoxib, SC-57666, DuP-697, parecoxib sodium, JTE-522, valdecoxib, SC-58125, etoricoxib, RS-57067, L-748780, L-761066, APHS, etodolac, meloxicam, S-2474, and mixtures thereof; glucocorticoids, such as hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, and combinations thereof.

Combinations of active agents, including telomerase inhibitor(s) of the invention, may be synergistic.

In one embodiment, the telomerase inhibitor is administered together with an inhibitor of NFκB. A “NFκB inhibitor” includes any molecule which decreases the activity of the NFκB or decreases the protein level of the NFκB. Thus, a NFκB inhibitor can be a molecule which decreases activity of the NFκB, for example by interfering with interaction of the NFκB with another molecule, e.g., its substrate. It can also be a molecule which decreases expression of the gene encoding the NFκB. The NFκB inhibitor may be a “direct inhibitor” or an “indirect inhibitor.”

The NFκB inhibitor can also be selected from the group consisting of interfering nucleic acid agent (such as a dsRNA or an antisense RNA or a ribozyme), an antibody, a small inorganic molecule, and PNA, as discussed above. In one example, the NFκB inhibitor is a dsRNA selected from the group consisting of shRNA, siRNA, and miRNA.

In one embodiment, the NFκB inhibitor is a dsRNA such as a shRNA.

In one embodiment, the NFκB inhibitor is a dsRNA, such as shRNA directed against p65 (such as sh-p65).

In one example, the shRNA is shRelA comprising SEQ ID NO: 54 (AGCCATTAGCCAGCGAATC). In one example, the shRNA is shRelA consisting of SEQ ID NO: 0.54 (AGCCATTAGCCAGCGAATC).

The term “shRNA” refers to a single strand RNA of about 10 to about 100 nucleotides, about 20 to about 100 nucleotides, about 22 to about 100 nucleotides, about 30 to about 100 nucleotides, about 40 to about 100 nucleotides, or about 50 to about 100 nucleotides, that forms a stem-loop structure in a cell, and which contains a loop region of about 5 to about 30 nucleotides, long complementary RNAs of about 15 to about 50 nucleotides at both sides of the loop region (which form a double-stranded stem by base pairing between the complementary RNAs), and additional 1 to about 500 nucleotides, about to about 500 nucleotides, about 100 to about 450 nucleotides, about 150 to about 400 nucleotides, or about 200 to about 350 nucleotides, included before and after each complementary strand forming the stem. shRNA is typically transcribed by RNA polymerase in a cell, and subsequently cleaved in the nucleus by Drosha. The cleaved shRNA is exported from the nucleus to cytosol, and further cleaved in the cytosol by Dicer. Like siRNA, shRNA binds to the target mRNA in a sequence specific manner, thereby cleaving and destroying the target mRNA, and thus suppressing expression of the target mRNA.

In some embodiments, the shRNA may include nucleic acids that also contain moieties other than ribonucleotide moieties, including, but not limited to, modified nucleotides, modified internucleotide linkages, non-nucleotides, deoxynucleotides, and analogs thereof. Within any shRNA, preferably a plurality and more preferably all nucleotides are ribonucleotides.

Suitable shRNA sequences for the knock down of a given target gene can readily be determined by a person skilled in the art. For example, suitable shRNA may be prepared from the microRNA-derived sequence, such as for example, mir-30-derived sequence.

The NFκB inhibitor can include, but is not limited to p65 shRNA (sc-29410-SH); sc-3060 (sequence: AAVALLPAVLLALLAPVQRKRQKLMP, SEQ ID NO: 47); 2-(1,8-naphthyridin-2-yl)-Phenol; 5-Aminosalicylic acid; BAY 11-7082; BAY 11-7085; CAPE (Caffeic Acid Phenethylester); Diethylmaleate; IMD 0354; Lactacystin; MG-132 [Z-Leu-Leu-Leu-CHO]; parthenolide; phenylarsine oxide; PPM-18; PyrrolidinedithiocarbaMic acid ammonium salt; (E)-3-(4-methylphenylsulfonyl)-2-propenenitrile; tetrahydrocurcuminoids; sulfasalazine; sulindac; clonidine; helenalin; wedelolactone; pyrollidinedithiocarbamate (PDTC); Calbiochem IKK-2 inhibitor VI; or Calbiochem IKK inhibitor III (BMS-345541).

In one embodiment, the NFκB inhibitor is an antibody directed against p65 (such as anti-p65).

The inhibitor compound(s) of the invention may also be present as suitable salts, including pharmaceutically acceptable salts. By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art. For instance, suitable pharmaceutically acceptable salts of compounds according to the present invention may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the invention.

The telomerase inhibitor(s) may be used in a combination therapy with one or more therapeutic agents to treat an inflammatory disease and/or cancer. For example, the telomerase inhibitor(s) may be used in a combined, separate, sequential or simultaneous administration with one or more NFκB inhibitor, or one or more anti-inflammatory drug, or one or more anti-cancer drug, as described herein. In one embodiment, such administration comprises co-administration of these therapeutic agents in a substantially simultaneous manner, for example in a single capsule having a fixed ratio of active ingredients or in multiple, separate capsules for each active ingredient. In another embodiment, such administration comprises use of each type of therapeutic agent in a sequential manner. In either case, the treatment regimen will provide beneficial effects of the drug combination in treating the disease or conditions described herein.

Convenient modes of administration include injection (subcutaneous, intravenous, etc.), oral administration, inhalation, transdermal application, topical creams or gels or powders, or rectal administration. Depending on the route of administration, the formulation and/or compound may be coated with a material to protect the compound from the action of enzymes, acids and other natural conditions which may inactivate the therapeutic activity of the compound(s). The compound(s) may also be administered parenterally or intraperitoneally.

The pharmaceutical compositions containing the inhibitor compound(s) can also include at least one pharmaceutically acceptable excipient. The use of such excipients for pharmaceutically active substances is well known in the art. Except insofar as any conventional excipient is incompatible with the inhibitor compound(s), use thereof in the therapeutic compositions and methods of treatment and prophylaxis is contemplated.

In one embodiment, the compound(s) of the invention may be administered orally, for example, with an inert diluent or an assimilable edible carrier. The compound(s) and other ingredients may also be enclosed in a hard or soft shell gelatin capsule, compressed into tablets, or incorporated directly into an individual's diet. For oral therapeutic administration, the compound(s) may be incorporated with excipients and used in the form of ingestible tablets, buccal tablets, troches, capsules, elixirs, suspensions, syrups, wafers, and the like. The excipients may include: a binder such as gum gragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavouring agent such as peppermint, oil of wintergreen, or cherry flavouring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the analogue, sucrose as a sweetening agent, methyl and propylparabens as preservatives, a dye and flavouring such as cherry or orange flavour. Any material used in preparing any dosage unit form should be pharmaceutically pure and substantially non-toxic in the amounts employed.

Suitably, such compositions and preparations may contain at least 1% by weight of active compound. The percentage of the active compound(s) in pharmaceutical compositions may, of course, be varied and, for example, may conveniently range from about 2% to about 90%, about 5% to about 80%, about 10% to about 75%, about 15% to about 65%; about 20% to about 60%, about 25% to about 50%, about 30% to about 45%, or about 35% to about 45%, of the weight of the dosage unit. The amount of compound in therapeutically useful compositions is such that a suitable dosage will be obtained.

Parenteral compositions may be formulated in dosage unit form for ease of administration and uniformity of dosage. “Dosage unit form” as used herein refers to physically discrete units suited as unitary dosages for the individual to be treated; each unit containing a predetermined quantity of compound(s) is calculated to produce the desired therapeutic effect in association with the required pharmaceutical carrier.

Another form of a pharmaceutical composition is a dosage form formulated as enterically coated granules, tablets or capsules suitable for oral administration.

Also included in the scope of this invention are delayed- or sustained-release formulations.

Compounds of the invention may also be administered in the form of a “prodrug”. A prodrug is an inactive form of a compound which is transformed in vivo to the active form. Suitable prodrugs include esters, phosphonate esters etc., of the active form of the compound.

In one embodiment, the compound may be administered by injection. In the case of injectable solutions, the excipient may be a carrier such as a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, it may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminium monostearate and gelatin.

Sterile injectable solutions can be prepared by incorporating the compound(s) in the required amount in an appropriate solvent with one or a combination of ingredients enumerated above, as required, followed by filtered sterilisation. Generally, dispersions are prepared by incorporating the compound(s) into a sterile vehicle which contains a basic dispersion medium and the required other ingredients from those enumerated above.

The pharmaceutical composition may further include a suitable buffer to minimise acid hydrolysis. Suitable buffer agent agents are well known to those skilled in the art and include, but are not limited to, phosphates, citrates, carbonates and mixtures thereof.

Single or multiple administrations of the pharmaceutical compositions according to the invention may be carried out. One skilled in the art would be able, by routine experimentation, to determine effective, non-toxic dosage levels of the compound and/or composition of the invention and an administration pattern which would be suitable for treating the diseases and/or infections to which the compounds and compositions are applicable.

Further, it will be apparent to one of ordinary skill in the art that the optimal course of treatment, such as the number of doses of the compound or composition of the invention given per day for a defined number of days, can be ascertained using convention course of treatment determination tests.

Generally, an effective dosage per 24 hours may be in the range of about 0.0001 mg to about 1000 mg per kg body weight; suitably, about 0.001 mg to about 750 mg per kg body weight; about 0.01 mg to about 500 mg per kg body weight; about 0.1 mg to about 500 mg per kg body weight; about 0.1 mg to about 250 mg per kg body weight; or about 1.0 mg to about 250 mg per kg body weight. More suitably, an effective dosage per 24 hours may be in the range of about 1.0 mg to about 200 mg per kg body weight; about 1.0 mg to about 100 mg per kg body weight; about 1.0 mg to about 50 mg per kg body weight; about 1.0 mg to about 25 mg per kg body weight; about 5.0 mg to about 50 mg per kg body weight; about 5.0 mg to about 20 mg per kg body weight; or about 5.0 mg to about 15 mg per kg body weight.

Alternatively, an effective dosage may be up to about 500 mg/m². For example, generally, an effective dosage is expected to be in the range of about 25 to about 500 mg/m², about 25 to about 350 mg/m², about 25 to about 300 mg/m², about 25 to about 250 mg/m², about 50 to about 250 mg/m², and about 75 to about 150 mg/m².

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes).

An inflammatory disease or condition that may be treated using the method disclosed herein can be selected from the group consisting of an inflammatory disease of the joints, an inflammatory disease of the skin, an inflammatory disease of the eyes, an inflammatory disease of the peripheral or central nervous system, an inflammatory disease of the airways or the lung, and an inflammatory disease of the gastrointestinal tract.

In one embodiment, the inflammatory disease is a disease or condition selected from the group consisting of mild cognitive impairment, rheumatoid arthritis, atherosclerosis, restenosis, pancreatitis, sepsis and peritonitis.

Preferential mention should be made of the prevention and treatment of diseases of the peripheral or central nervous system. Examples of these include depression, bipolar or manic depression, acute and chronic anxiety states, schizophrenia, Alzheimer's disease, Parkinson's disease, acute and chronic multiple sclerosis or acute and chronic pain as well as injuries to the brain caused by stroke, hypoxia or craniocerebral trauma.

Particular mention should be made of the prevention and treatment of diseases of the airways which are accompanied by increased mucus production, inflammations and/or obstructive diseases of the upper and lower respiratory tract, including the lungs. Examples include acute, allergic or chronic bronchitis, chronic obstructive bronchitis (COPD), coughing, pulmonary emphysema, allergic or non-allergic rhinitis or sinusitis, chronic rhinitis or sinusitis, asthma, alveolitis, idiopathic pulmonary fibrosis, fibrosing alveolitis, Crohn's disease, ulcerative colitis, Farmer's disease, hyperreactive airways, infectious bronchitis or pneumonitis, paediatric asthma, bronchiectases, pulmonary fibrosis, ARDS (acute adult respiratory distress syndrome), bronchial oedema, pulmonary oedema, bronchitis, pneumonia or interstitial pneumonia triggered by various causes, such as aspiration, inhalation of toxic gases, or bronchitis, pneumonia or interstitial pneumonia as a result of heart failure, irradiation, chemotherapy, cystic fibrosis or mucoviscidosis, or alphal-antitrypsin deficiency.

Also deserving mention is the treatment of inflammatory diseases of the gastrointestinal tract. Examples include acute or chronic inflammatory changes in gall bladder inflammation, Crohn's disease, ulcerative colitis, inflammatory pseudopolyps, juvenile polyps, colitis cystica profunda, pneumatosis cystoides intestinales, diseases of the bile duct and gall bladder, e.g. gallstones and conglomerates.

In one embodiment, the inflammatory disease or condition is one that is mediated by NFκB. Such NFκB-mediated diseases include diseases in which multiple biological pathways and/or processes in addition to NFκB-mediated processes contribute to the disease pathology. A NFκB-mediated disease may be completely or partially mediated by modulating the activity or amount of NFκB. Exemplary diseases that may be completely or partially mediated by NFκB include, but are not limited to, muscular dystrophy, arthritis, traumatic brain injury, spinal cord injury, sepsis, rheumatic disease, cancer atherosclerosis, type 1 diabetes, type 2 diabetes, leptospiriosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, ischemia/reperfusion, stroke, cerebral aneurysm, angina pectoris, pulmonary disease, cystic fibrosis, acid-induced lung injury, pulmonary hypertension, asthma, chronic obstructive pulmonary disease, Sjogren's syndrome, hyaline membrane disease, kidney disease, glomerular disease, alcoholic liver disease, gut diseases, peritoneal endometriosis, skin diseases, nasal sinusitis, mesothelioma, anhidrotic ecodermal dysplasia-ID, behcet's disease, incontinentia pigmenti, tuberculosis, asthma, crohn's disease, colitis, ocular allergy, appendicitis, paget's disease, pancreatitis, periodonitis, endometriosis, inflammatory bowel disease, inflammatory lung disease, silica-induced diseases, sleep apnea, AIDS, HIV-1, autoimmune diseases, antiphospholipid syndrome, lupus, lupus nephritis, familial mediterranean fever, hereditary periodic fever syndrome, psychosocial stress diseases, neuropathological diseases, familial amyloidotic polyneuropathy, inflammatory neuropathy, parkinson's disease, multiple sclerosis, alzheimer's disease, amyotropic lateral sclerosis, huntington's disease, cataracts, and hearing loss.

Preferential mention should also be made of the treatment of cancers. Cancers that may be treated using the method disclosed herein can be selected from the group consisting of acute and chronic leukaemia (such as acute lymphatic leukaemia, acute lymphocytic leukaemia, acute myeloid leukaemia, chronic lymphatic leukaemia, chronic lymphocytic leukaemia, and chronic myeloid leukaemia), bone tumors (such as osteosarcoma), all types of glioma (such as oligodendroglioma and glioblastoma), breast cancer, colon cancer, lung cancer, prostate cancer, and stomach cancer.

In one example, the treatment of cancer is excluded.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 demonstrates the functional intersection between telomerase and NFκB signalling.

A. Cell proliferation assay. A2780cp cells were infected with viruses expressing GFP (Cntrl) or TERT+Terc (TT) and sh-control (sh-cntrl) or sh-p65 (sh-p65) individually or together as indicated and used for all the assays. 20,000 cells were plated in 6-well plates and counted every alternate day.

B. Control infected (Cntrl) or p65 expressing cells (p65) were transfected with si-hTERT (si-hTERT) or si-Cntrl (si-Cntrl) as indicated. For cell proliferation assay 20,000 cells were plated in 6-well plates and counted every alternate day and cell number plotted.

C-D. Cell death assay: 70,000 cells were plated in 12-well plates in duplicates for each condition. Cells were treated with Doxorubicin as indicated. 48 hrs after treatment, all cells (adherent and floating) were collected, mixed with trypan blue and counted by haemocytometer. The number of dead cells is represented as percentage of the total cells.

(E-H.) A2780cp cells were infected with viruses expressing GFP (Cntrl) or TERT+Terc (TT) and sh-control (sh-cntrl), sh-hTERT (sh-hTERT) or sh-p65 (sh-p65) individually or together as indicated and used for all the assays. E. Colony formation assay: 1000 cells per condition were plated in 0.60 mm plates in RPMI supplemented with 10% FBS for 2 days followed by RPMI with 3% FBS for an additional 10 days. Colonies were stained with crystal violet after 12 days. Number of colonies was counted from duplicate plates. F. Quantitation of colonies formed in E. G. Colony formation assay: 1000 cells per condition were plated in 60 mm plates in RPMI supplemented with 10% FBS for 2 days followed by RPMI with 3% FBS for an additional 10 days. Colonies were stained with crystal violet after 12 days. Number of colonies was counted from duplicate plates. H. Quantitation of colonies formed in G.

(I-K.) In vivo tumor formation assay: 10⁶ A2780cp cells with indicated expression conditions were injected subcutaneously in 8-10 week old nude mice (N=3). Tumors were visualised at 4 weeks, resected and weighed. I. Representative photographs of tumor bearing mice sacrificed 4 weeks after injections. J. Representative photograph of excised tumors from mice. K. Weights of tumors excised.

(L-N.) In vivo tumor formation assay: 10⁶ A2780cp cells with indicated expression conditions were injected subcutaneously in 8-10 week old nude mice (n=3). Tumors were visualised at 4 weeks, resected and weighed. L. Representative photographs of mice bearing tumors. M. Representative photograph of resected tumors. N. Weights of tumors excised.

FIG. 2 demonstrates that telomerase regulates NFκB dependent gene expression.

A. Telomere restriction fragment length analysis (TRF) performed for A2780cp cells infected with the indicated viruses and controls. Genomic DNA was prepared after 2 weeks of culture followed by restriction digestion and southern blotting.

B. NFκB dependent luciferase reporter assay: 293T cells were co-transfected with TT or GFP control plasmids along with Luciferase reporter plasmids. Cells were treated with 10 ng/ml TNFα followed by measurement of luminescence in lysates after 16 hrs. Expression of firefly luciferase reporter was normalized to Renilla luciferase expression. C. A2780cp cells were infected with viruses expressing GFP/sh-control, TT and sh-p65 individually or together as indicated and cultured for 2 weeks followed by mRNA extraction. Levels of relative mRNA expression of indicated genes are shown.

D. Cells were transfected with TT or GFP (Cntrl) plasmids as indicated for 48 hrs. Cells were treated with 10 ng/ml TNFα for 1 hr followed by mRNA extraction and quantitative PCR. Levels of relative mRNA expression for the indicated genes are shown.

E. Cells were transfected with siRNA for hTERT (si-hTERT) or scrambled controls (si-Cntrl) as indicated. 65 hrs later cells were treated with 10 ng/ml TNFα for 1 hr followed by mRNA extraction and quantitative PCR. Levels of relative mRNA expression for the indicated genes are shown.

F. Cells were treated with 2 μM MST-312 or DMSO as indicated for 48 hrs. Cells were treated with 10 ng/ml TNF for 1 hr followed by mRNA extraction and quantitative PCR. Levels of relative mRNA expression for the indicated genes are shown.

G. Cell death assay: 70,000 cells were plated in 12-well plates in duplicates for each condition. Cells were treated with 2 μM MST-312 prior to TNFα dose response. The treatments were done as indicated and all cells (adherent and floating) were collected, mixed with trypan blue and counted by haemocytometer. The number of dead cells is represented as percentage of the total cells.

FIG. 3 demonstrates that telomerase null mice display defective NFκB signalling.

A. Telomerase deficient mice (mTerc^{-/-}) are resistant to endotoxic shock. Matched cohorts (n=10) of the indicated genotypes of mice were sensitized with GalN (700 μg/kg) followed by LPS (50 μg/kg) challenge and hourly monitoring for death. The survival curve is presented as percentage survivors over time post challenge.

B. Mice deficient for either the telomerase RNA component mTerc or the catalytic component mTERT are resistant to LPS challenge. Matched wild-type (n=8), mTerc^{-/-} (n=6) and mTERT^{-/-} (n=6) cohorts were sensitized with GalN followed by LPS challenge and hourly monitoring for death. Bars indicate percent surviving animals over time post challenge.

C. mTerc^{-/-} MEFs are defective for NFκB dependent gene expression. mTerc^{-/-} and mTerc^{+/-} MEFs were derived and stimulated with TNFα for 2 hrs and analyzed for gene expression. Indicated genes were measured by quantitative PCR analysis of the extracted RNA from these MEFs. The results shown are representative of 5 independent pairs of knock out and control MEFs.

D. mTERT MEFs are defective for NFκB dependent gene expression. mTERT^{+/+} and mTERT^{-/-} MEFs were derived and then stimulated with TNFα for 2 hrs. Relative mRNA expressions for the indicated genes are shown.

E. Telomere restriction fragment length analysis for 2 independent pairs of mTerc^{-/-} and mTerc^{+/-} MEFs.

FIG. 4 shows that telomerase is recruited to selective NFκB target gene promoters.

A. Telomerase association with p65 was analyzed using immunoprecipitation with anti-hTERT antibody in cells treated with TNFα for the indicated durations.

B. HeLa cells treated with 10 ng/ml TNFα for the indicated durations were fractionated to obtain nuclear and cytoplasmic extracts. Immunoprecipitation using 1 mg protein with hTERT antibody was done for each fraction. The indicated proteins were probed by western blotting. p65 blots were stripped and reprobed for TRF2 as loading control for nuclear fraction. GAPDH served as loading control for cytosolic fraction.

C. Cells were treated with TNFα for the indicated time. Immunoblots of indicated proteins were performed for co-immunoprecipitations with anti-p65 antibody from nuclear and cytosolic extracts.

D. Chromatin immunoprecipitation showing recruitment to NFκB promoters. HeLa cells treated with either DMSO or 2 AM MST-312 were stimulated with 10 ng/ml of TNFα for 1 hr. The cells were lysed and ChIP was performed using the indicated antibodies. PCR from ChIP eluates of indicated conditions followed by resolution on a 1.5% agarose gel shows binding to promoters of indicated genes.

E. Quantitation of the hTERT binding to the promoters in A2780cp cells. Bars represent fold change in binding compared to control based on quantitative real-time PCR. F. ChIP-western analysis of immunoprecipitated complexes from cells treated with DMSO or 2 μM MST-312 and stimulated with TNFα as indicated.

G. Re-ChIP on A2780cp cells: Eluates from p65 ChIP were used to re-ChIP using hTERT or IgG antibodies. The eluates from re-ChIP were used for quantitative PCR using promoter specific primers. Bars represent fold enrichment of binding over IgG controls.

H. Cells were treated with 2 μM MST-312 or DMSO control followed by 10 ng/ml TNFα treatment for 1 hr. The cells were lysed and ChIP was performed using the antibodies indicated. PCR from ChIP eluates of indicated conditions followed by resolution on a 1.5% agarose gel shows binding to promoters of indicated genes.

I. Bars represent quantification of p65 binding to a subset of NFκB genes by quantitative PCR of ChIP eluates and controls after the indicated treatments.

J. HeLa cells transfected with 80 nM si-hTERT or si-Cntrl for 65 hrs. Subsequently, these cells were treated with 10 ng/ml of TNFα for 1 hr followed by ChIP with the indicated antibodies. PCR from ChIP eluates of indicated conditions followed by resolution on a 1.56 agarose gel shows binding to promoters of indicated genes.

FIG. 5 shows reduction in TNFα-induced genome-wide p65 occupancy due to telomerase inhibition.

A. Number of p65 ChIP-seq peaks upon TNFα and MST-312 treatments. TNFα treatment significantly induces p65 binding, but the number of p65 bound regions is reduced in the presence of MST-312. Peaks are considered overlapping if they are within 500 bps. p65 peaks, which are overlapping in presence and absence of MST-312, are referred as the “common” peaks (N=624), and peaks, which are lost or gained due to MST treatment, are named as “TNF unique” (N=647) and “TNF&MST unique” (N=228) peaks, respectively.

B. The ChIP-seq peaks in common between the TNFα treated HeLa cells before and after MST-312 (“common peaks w/o MST”: without MST-312, and “common peaks with MST”: with MST-312 treatment) show greater p65 occupancy than those peaks unique to TNFα treatment (“TNFα unique”) or unique to TNFα+MST-312 (“TNFα&MST unique”). These “unique” peaks are predominantly weak binding sites and may represent less stable binding sites.

C. MST-312 treatment reduces p65 occupancy in the “common” sites defined by the intersect region depicted in (A). There is a statistically significant shift in the distribution of p65 occupancy to less binding in the presence of MST-312 (modal peak without MST=9.67 shown in red, and with MST=5.76 shown in green, P=1.111e-13).

D. TNFα treatment significantly increases p65 occupancy at “common” p65 binding sites (compare “W/O TNF” line vs. “with TNF” lines and “W/O TNF” line vs “with TNF&MST” line). The effect of MST-312 on p65 binding is significant but quantitatively limited (compare “with TNF&MST” line vs “with TNF” line). The abscissa is tag density as the number of tags per 100 bp, the ordinate represents the distance in base pairs from the center of each p65 binding sites.

E. The quantitative reduction in p65 binding after MST-312 exposure appears to be in 13.1% of the common peaks. Significant fold change is considered to be either 1.5 fold (log 2[FC]=+/−0.585) increase or decrease from TNFα treatment alone. A 2 fold (log 2[FC]=1) reduction in p65 occupancy at the IL-6 promoter can be seen.

FIG. 6 shows that enhanced NFκB binding to IL6 promoter is dependent on telomerase.

A. TNFα treatment stimulates p65 binding at IL6 promoter (“p65_Input”=input DNA, “p65_DMSO”=DMSO treatment p65 ChIP, “p65 TNF”=TNFα treatment p65 ChIP). MST-312 reduces TNFα induced p65 occupancy (“p65_MST_TNF”=TNFα +MST-312 treatment, vs. “p65 TNF”).

B. Sequences of oligonucleotide probes used for Electrophoretic mobility shift assay (EMSA). Probes: NFκB consensus (double stranded, 5′-TCA ACA GAG GGG ACT TTC CGA GAG GCC-3′, SEQ ID NO: 39), IL6A (double stranded, ST-ACT GGG AGG ATT CCC AAG GGG TCA ATT GGG AGA-3′, SEQ ID NO: 40), IL6B (double stranded, 5′-ACT GGG AGG ATT CCC AAG GGG TCAA-3′, SEQ ID NO: 41) and Oct-1 (double stranded, 5′-TGT CGA ATG CAA ATC ACT AGA A-3′, SEQ ID NO: 46) radiolabeled probes.

(C-F.) EMSA was performed using nuclear extracts from cells treated under specified conditions. C. EMSA for Oct-1 control and NFκB consensus probes using extracts from si-Cntrl or si-hTERT cells. D. EMSA for the endogenous IL6 promoter based oligos with (IL6A) or without (IL6B) a putative TERT binding site run on the same gel. E. EMSA supershift analysis was done with the indicated antibodies on nuclear extracts from cells stimulated with TNFα on the synthetic NFκB consensus sequence. F. EMSA supershift analysis was done with the indicated antibodies on the endogenous ILEA promoter sequence. Treatment and antibodies in each lane are indicated as per lane numbers.

G. IL6 expression in primary patient samples after treatment with 0.5μ MST-312 for 48 hrs. Expression levels are shown as percentage expression of IL6 in MST-312 treated samples compared to the matched DMSO treatment for each individual patient line (X-axis). ND—not detectable.

H. A model based on our studies. In resting cells NFκB dependent gene transcription regulates proliferation, resistance to apoptosis and innate immune responses. This pathway is rapidly turned off. However, in cancer cells, which require sustained and enhanced activity of NFκB target genes, reactivated hTERT (a NFκB target gene), a limiting factor for telomerase activity, stabilizes p65 on a subset of target gene promoters and increases expression of NFκB target genes which drive invasion, cellular proliferation, resistance apoptosis, all necessary hallmarks of cancer. In addition, these cancer cells secrete cytokines attracting macrophages that produce more NFκB activating cytokines. Hence, this feed forward pathway sustains levels of NFκB as well as telomerase at a critical level such that its telomere dependent and independent activities aid in the process of transformation.

FIG. 7 shows the functional intersection between telomerase and NFκB signalling in cancer cells.

(A-E.) Cells expressing GFP (Cntrl) or TT and IκBαM mutant individually or together as indicated and used for all the assays. A. Cell proliferation assay. B. Cell death assay. C. Colony formation assay. D. Quantitation of colonies formed. E. Invasion assay was performed using the Millipore QCM invasion assay kit. Number of invaded cells was evaluated and represented as relative fluorescence units (RFU).

F. Colony formation assay: A2780cp cells were infected with p65 expressing or GFP (Cntrl) virus followed by plating 1000 cells per condition. The cells were then treated with 2 μM MST312 or DMSO control for 2 weeks followed by staining with crystal violet after 12 days. Number of colonies was counted from duplicate plates.

G. Quantitation of colonies formed.

H. Colony formation assay: 1000 cells per condition were plated in 35 mm plates followed by staining with crystal violet after 12 days. Number of colonies was counted from duplicate plates. Cells were treated with indicated doses of MST-312 and DMSO with or without 5 μM IKK inhibitor (SC514).

I. Quantitation of colonies forme.

FIG. 8 demonstrates that telomerase and NFκB cross-talk in different cancer cells.

A. HepG2 cells were infected with GFP control (Cntrl) or p65 expressing virus followed by transfection with control si RNA (si-Cntrl) or si-hTERT (si-hTERT) as indicated. Cell proliferation assay was performed as indicated before.

B HepG2 cells expressing GFP (Cntrl) or Terc+TERT (TT) and sh-p65 (sh-p65) or sh-control (shCntrl) individually or together as indicated were used in cell proliferation assay as previously described.

C. Cell death assay using cells infected with the indicated viral vectors either alone or in combination.

D. HepG2 cells expressing control (Cntrl) or Terc TERT (TT) either alone or in combination with IκBαM (IκBαM) were used in invasion assay using the Millipore QCM invasion assay kit. Number of invaded cells was evaluated and represented as relative fluorescence units (RFU). (E-F.) MCF7 cells expressing GFP (Cntrl) or TT and IκBαM mutant individually or together as indicated and used for E. Cell death assay. F. Invasion assay which was performed using the Millipore QCM invasion assay kit. Number of invaded cells was evaluated and represented as relative fluorescence units (RFU).

FIG. 9 shows that telomerase does not regulate cytosolic NFκB signalling.

A. HeLa Cells infected with GFP control virus (Cntrl) or TT (Lanes 1 and 2). Telomerase null VA13 cells were infected with control virus or TT (Lanes 3 and 4). HeLa cells were treated with 80 nM hTERT siRNA or scrambled control for 65 hrs (Lanes 5 and 6). Levels of hTERT, IKK and p65 were analysed 65 hrs after infections or transfections.

B. 293T cells were transfected with TT or GFP control plasmids for 48 hrs. Cells were then treated with 10 ng/ml TNFα for the indicated durations. Phosphorylated and total amounts of proteins were-analysed by immunoblotting for the indicated proteins over the time course.

C. BJ fibroblasts and BJ-hTERT fibroblasts were treated with 10 ng/ml TNFα for the indicated durations. Immunoblots were performed for the indicated proteins.

FIG. 10 shows the effect of telomerase dose on NFκB dependent gene expression. 293T cells were transfected with the indicated amounts of TT plasmid. 48 hrs post transfection, cells were treated with 10 ng/ml TNFα followed by quantification of relative mRNA expression of TNF (A) and IκBα (B) by qPCR.

FIG. 11 shows that telomerase promotes NFκB dependent transcription in different cell lines.

A. VA13 cells were infected with virus expressing TT or GFP controls. Graphs represent relative mRNA expression levels of the indicated genes upon stimulation with 10 ng/ml TNFα for 1 hr.

B. Total RNA was extracted from VA13 and Wi38 cells after stimulation with 10 ng/ml TNFα for 1 hr. Relative mRNA expression levels of the indicated genes was quantified by qPCR.

C. Relative mRNA expression levels of indicated genes TNF and IL6 from BJ and BJ-hTERT fibroblasts treated with 10 ng/ml TNFα for 1 hr.

FIG. 12 shows that MST-312 does not affect NFκB signalling directly. VA13 cells were treated with 2 μM MST-312 or DMSO for 24 hrs followed by stimulation with 10 ng/ml TNFα for the indicated durations.

A. Phosphorylated and total amounts of proteins were analysed by immunoblotting for the indicated proteins over the time course.

B. Relative mRNA expression levels of indicated genes after stimulation with TNFα for 1 hr.

FIG. 13 shows the effect of telomerase components on NFκB dependent gene expression.

A. HeLa cells were infected with virus expressing TT, hTERT alone, hTERT-DN (dominant negative), hTerc alone or GFP control for 7 days followed by TNFα treatment for 1 hr. Relative mRNA expression levels of the indicated genes were quantified by qPCR.

B. HeLa cells were infected with shRNA to hTERT, hTerc or scrambled control for 7 days followed by TNFα treatment for 1 hr. Relative mRNA expression levels of the indicated genes were quantified by qPCR.

FIG. 14 shows that telomerase null MEFs have defective NFκB signalling.

A. Relative mRNA expression levels of indicated genes after 1 μg/ml LPS treatment for 1 hr in mTerc^{-/-} and mTerc^{+/-} MEFs.

B. Relative mRNA expression levels of indicated genes after 10 ng/ml IL-1 treatment for 1 hr in mTerc^{-/-} and mTerc^{+/-} MEFs.

C. Mouse cohorts of indicated genotypes were injected with 10⁶ Listeria monocytogenes bacterial challenge. 3 days after infections, the spleens were harvested and dissociated in 10 ml PBS. The graph shows the number of bacteria recovered from individual animal spleens as measured by serial plating of extracted bacteria; horizontal bars demonstrate mean values for each group.

FIG. 15 shows that p65 binds to hTERT in a stimulus dependent manner in primary human mammary epithelial cells. HMEC-hTERT fibroblasts were treated with TNFα and co-immunoprecipitations were done with anti-p65 antibody from nuclear and cytosolic extracts. Immunoblots shown for indicated proteins.

FIG. 16 shows the functional interaction between NFκB p65 and hTERT in IMR90 cells.

A. Immunoblots as indicated for co-immunoprecipitation from IMR90-hTERT cells with anti-hTERT antibody after TNFα treatment.

B. IMR90-hTERT cells were infected with GFP Cntrl or sh-p65 virus followed, by treatment with Doxorubicin doses as indicated. 48 hrs later, percentage of cell death was measured by trypan blue assay.

C. IMR90-hTERT cells were treated with 2 μM MST-312 followed by treatment with Doxorubicin doses as indicated. 48 hrs later, percentage of cell death was measured by trypan blue assay.

D. Chromatin immunoprecipitation showing recruitment to NFκB promoters. IMR90-hTERT cells were treated with 10 ng/ml of TNFα for 30 min.

E. Quantification of hTERT binding on IL6 and TNF promoters.

FIG. 17 shows that telomerase does not affect TNF dependent nuclear translocation of p65.

A. HeLa cells were treated with 2 μM MST-312 (lanes 3-4), DMSO (lanes 1-2) or transfected with si-hTERT, si-Cntrl (Lanes 5-8) or infected with TT, Cntrl virus (Lanes 9-12) as indicated. 48 hrs later, cells were stimulated with 10 ng/ml TNFα for 30 min followed by fractionation of nuclear and cytosolic fractions. Immunoblot shows nuclear fractions with indicated treatments. p65 blots were stripped and reprobed for TRF2.

B. A2780cp cells were infected with the indicated viruses (as indicated in the bottom of the panels). Relative mRNA levels of the indicated genes (as indicated on the top of the panels) were measured by quantitative PCR 2 weeks after infection.

FIG. 18 shows that p65 primarily binds to inter- and intragenic regions.

A. Gene model based on peak position relative to TSS. Gene regulatory regions are defined as proximal promoter (within 2.5 up- or downstream of TSS), distal promoter (within 20 kB upstream of TSS), 3′ UTR (within 2.5 kB downstream of gene body), exon, intron, and intergenic region (outside gene body, promoter regions and 3′UTR). B. Association of p65 peaks to the gene regulatory regions. P65 peaks are overrepresented in intronic and intragenic regions. The profile of association remains same in presence or absence of MST-312.

FIG. 19 shows that p65 motif is the strongest motif for each subset of binding sites on ChIP seq. A screen shot from CENTDIST motif enrichment tool is shown. p65 motif is identified as the strongest motif for common peaks (A), TNF-unique peaks (B), and TNF&MST-unique peaks (C). In all groups, p65 had highest score among all motifs, motif distribution is centered around peak summit, and percentage of peaks having the motif sequence decreases as peaks get weaker. The rank, score and distribution are shown. Enrichment analysis confirms that each group contains peaks with good quality.

FIG. 20 shows the Differential Motif Enrichment Analysis between telomerase sensitive and insensitive p65 binding sites.

A. Motif analysis for PWM (position weight matrix) using telomeric repeats shown from TRF1 binding motif was used for the analysis since there is no known PWM for TERT binding motif.

B. There is a statistically significant enrichment of telomeric repeat motif in TERT-dependent binding sites (plain line, n=85) compared to TERT-independent binding sites (dashed line, n=408). Telomeric repeats were scanned around +/−2500 bp of peak summit (FDR=0.001). The density of motif occurrence around peak center in each group of peaks is plotted. P-value is calculated based on binomial distribution.

FIG. 21 shows the prediction of biological processes that genes in proximity to p65 peaks are involved in. GREAT outcome for prediction of top 20 biological processes that common peaks are likely to be involved is shown. Most of the processes enriched for common peaks are related to inflammation.

FIG. 22 shows genome wide p65 binding sites. Common p65 binding sites between the TNFα and TNFα&MST-312 treatments are reduced after MST-312 treatment. Common binding sites which are reduced by at least 1.5 fold in presence of MST-312 are represented on a chromosome map (“TNF w/o MST”: without MST, “TNF with MST”: with MST, N=85). From the target genes tested in the functional assays, only IL6 and TNF are associated with a common p65 binding site exhibiting a decreased binding by minimum 1.5 fold.

FIG. 23 shows that telomerase regulates NFκB binding to IL8 and TNF promoters. A2780 ovarian cancer cells were treated with 10 ng/ml TNFα for 30 min and nuclear extracts were subject to EMSA to measure NFκB binding activity using (A) IL8A (wild type IL8 promoter with putative telomerase binding site) and IL8B (mutant IL8 promoter without putative telomerase binding site) and (B) TNFαA (wild type TNFα promoter with putative telomerase binding site) and TNFαB (mutant TNFα promoter without putative telomerase binding site) probes. An Oct-1 (C) probe served as an EMSA loading control.

FIG. 24 shows the details of patient samples. The table shows the diagnosis and cytogenetic analysis of the patient samples used in the study. Abbreviations used: ALL—Acute Lymphocytic Leukemia; AML—Acute Myeloid Leukemia; CML—Chronic Myeloid Leukemia; CML-BC=CML in blast crisis; CML-CP=CML in chronic phase; NA=Not available; ND=not done/not requested

### DETAILED DESCRIPTION OF DRAWINGS

### Examples

Non-limiting examples of the invention, including the best mode, and a comparative example will be further described in greater detail by reference to specific Examples, which should not be construed as in any way limiting the scope of the invention.

### Methods

### Cells and Reagents

Wild-type (WT), mTerc^{-/-}, mTERT^{-/-}, MEFs were derived by timed mating of mTerc^{+/-} or mTERT^{+/-} breeding pairs as described previously^{15,36,37,39,42}. Briefly, embryos were harvested at E13.5, internal organs removed and fibroblasts cultured in Dulbecco's modified eagle medium (DMEM) supplemented with 10% heat-inactivated fetal bovine serum (FBS), 2 mM L-glutamine, 2 mM sodium pyruvate, 2% β-mercaptoethanol and 1×PSF (penicillin, streptomycin and fungizone) at 37° C. with 10% CO₂. HeLa, 293T, MDA-MB-231 and MEFs were grown in Dulbecco's modified eagle medium (DMEM); A2780cp and MCF7 cells were grown in RPMI; both media were supplemented with 10% heat-inactivated fetal bovine serum (FBS), 2 mM L-glutamine, 2 nM sodium pyruvate and 1×PSF (penicillin, streptomycin and fungizone) at 37° C. with 0.5% CO₂. Antibodies against IKK (IKKα; Sc7182, Sc7218), IKK1/2 (IKKα/β; Sc7607), NEMO (IKKγ; Sc8330/AH00442), p65 (Sc8008; Sc372) and IκBα (Sc371), were from SantaCruz Biotechnology. hTERT specific antibody was obtained from Epitomits (Epitomics 1531-1) and Calbiochem. Antibodies against phosphorylated-p65 (Ser 536; A300-306A), phosphorylated-IκB (Ser 32), p100/p52 were from Cell Signaling Technology. TRF2-specific antibodies were from Millipore. All antibodies were used at 1:1000 dilution TNFα and IL-1α (Calbiochem) were used 10 ng/ml, whereas LPS (Sigma, L2654) was used at 1 μg/ml. MST-312 and DMSO were from Sigma.

Plasmid, shRNA/siRNA Transfection

Plasmids overexpressing telomerase holoenzyme or individual components were a gift from Shang Li and reported earlier^{86,87}. Plasmids expressing short hairpin RNA against human TERT have been previously described⁸⁶. Plasmids overexpressing p65, dominant negative IκBα mutant^{13,88} or shRNA to p65 have been described before. siRNA against hTERT or hTerc was obtained from Qiagen or Dharmacon. Control siRNA was from Qiagen (All Star Negative Control). Cells were transfected using either Lipofectamine LTX or Lipofectamine RNAiMax for 48-72 hr according to the manufacturer's instructions prior to use in experiments.

### Viruses

Lentiviruses and retroviruses were constructed and made as described¹³ previously.

### Cell Death Assay

Cells were plated 7×10⁴ per well of a 12-well plate in duplicates for 24 hrs. They were treated doxorubicin or TNFα for 24 and 48 hrs. Thereafter, all cells in the supernatant as well as adherent were collected from each well and resuspended in equal volume of 1×PBS. The cells were diluted 1:10 in PBS and equal volume of trypan blue dye was added to each sample. Thereafter total number of cells and total blue cells (dead cells that did not exclude the dye) were counted by haemocytometer. Cell death is presented as the percentage of dead cells compared to total cells for each well.

### Cell Proliferation

Cells previously infected with different viruses were plated in 6-well plates at 2×10⁴ cells per well in triplicates. Cells were counted every alternate day from 1 to 10 days.

### Invasion Assays

Invasion assays were performed according to manufacturer's instructions (Millipore QCM invasion assay). Briefly, after infection with indicated viruses, the cells were starved in media without FBS/growth factors for 18-24 hrs. A cell suspension containing 75,000 cells was loaded into the chamber inserts and incubated for 48 hrs. Invading cells on the bottom of the insert membrane were dissociated from the membrane with cell-detachment buffer. Cells were lysed and detected by CyQUANT GR dye (Molecular Probes).

### Teloblots

Genomic DNA was isolated from each individual cell pellet after indicated infections or transfections. 2 mg of genomic DNA was digested with HinfI/RsaI for 3-5 hours at 37° C. Thereafter, the digested. DNA is run on 0.6% agaroses gel in 1×TBE buffer gel over night at 80 volt. Following depurination and denaturation, DNA was transferred overnight and membranes probed with (CCCTAA)₄ radiolabelled probes.

### NFκB Luciferase Reporter Assay

For NFκB luciferase assay, 10000 HeLa or A549 cells were seeded in 24-well plates and transfected with Lipofectamine 2000. Cells were transiently transfected with 200 ng plasmids encoding NFκB luciferase reporter, 20 ng of pRL-CMV (Renilla luciferase) and 100 ng GFP or TT. 16-24 h after the transfection, cells were treated with 10 ng/mL TNFα for 6 h. For the luciferase assay, cells were lysed in reporter lysis buffer and activity was measured with the luciferase assay reagent (Promega) according to manufacturer's instructions. Relative luciferase activities are expressed as fold of activation over the activity of NFκB luciferase reporter alone and were calculated by dividing the values of firefly luciferase activity with the values for renilla luciferase activity. Three independent experiments were performed for each group.

### Western Blot Analysis

Total protein was extracted with Totex buffer (20 mM Hepes at pH 7.9, 0.35 M NaCl, 20% glycerol, 1% NP-40, 1 mM MgCl2, 0.5 mM EDTA, 0.1 mM EGTA, 50 mM NaF and 0.3 mM NaVO3) containing a mixture of protease inhibitors (Roche). Immunoblotting was performed with specific antibodies and visualized using ECL western blotting detection kit (Amersham Bioscience).

### Co-Immunoprecipitation

Cells were washed with ice-cold PBS and then lysed in a solution containing 10 mM Tris at pH 8, 170 mM NaCl, 0.5% NP40 and protease inhibitors for 30 min on ice. Cell lysates were removed by centrifugation and the supernatants were incubated with anti-p65 antibody overnight at 4° C. and with protein G-Sepharose for a further 2 hrs. Beads were washed four times with 1 ml of wash buffer (containing 200 mM Tris at pH 8.0, 100 mM NaCl and 0.5% NP-40). Bound proteins were eluted with SDS sample buffer and separated on NuPAGE Novex 4-12% Bis-Tris(Bis(2-hydroxyethyl)-amino-tris(hydroxymethyl)-methane) gels before Immunoblotting with specific antibodies.

### Nuclear Cytoplasmic Fractionation

The cells were harvested in ice-cold PBS and resuspended in hypotonic lysis buffer (10 mM HEPES pH7.9, 1.5 mM MgCl₂, 10 mM KCl, protease and phosphatse inhibitors) and incubated on ice for 4 min. They were, then spun down for 3 min at 4500 rpm and the cytoplasmic fraction aspirated to separate tubes. The pellet was washed with hypotonic buffer once. The nuclear fraction was then lysed in IP lysis (10 mM Tris at pH 8, 170 mM NaCl, 0.5% NP40 and protease inhibitors) buffer on ice for 30 min. The lysate was clarified by centrifugation and 1 mg of each fraction was used for corresponding IP reactions.

### Quantitative Real-Time PCR.

Total RNA was isolated using RNeasy Kit (Qiagen) according to the manufacturer's instructions. cDNA was prepared from 1-2 μg of RNA Superscript Vilo reverse transcriptase (Invitrogen). Real-time PCR reactions were performed in duplicates using SYBR GreenER (Invitrogen) according to the manufacturer's instructions. Cycles for SYBR GreenER PCRs: 7.5 min at 95° C. for the initial denaturation, followed by 40 cycles of 95° C. for 15 s and 60° C. for 30 s. Primer sequences are set out in the following Table:

Human

| | |
|---|---|
| hIL-8 FW | GCC AAC ACA GAA ATT ATT GTA AAG CTT (SEQ ID NO: 1) |
| hIL-8 RV | CCT CTG CAC CCA GTT TTC CTT (SEQ ID NO: 2) |
| hTNFa FW | TGG TAT GAG CCC ATC TAT CTG (SEQ ID NO: 3) |
| hTNFa RV | AGA CTC GGC AAA GTC GAG ATA (SEQ ID NO: 4) |
| hIKBa FW | CGC ACC TCC ACT CCA TCC (SEQ ID NO: 5) |
| IKBa RV | AGC CAT GGA TAG AGG CTA AGT GTA G (SEQ ID NO: 6) |
| BetaActin FW | GCC AAC CGC GAG AAG ATG A (SEQ ID NO: 7) |
| BetaActin RV | CCA TCA CGA TGC CAG TGG TA (SEQ ID NO: 8) |
| MCP1 FW | GCT CGC TCA GCC AGA TGC A (SEQ ID NO: 9) |
| MCP1 RV | GGA CAC TTA CTG CTG GTG ATT C (SEQ ID NO: 10) |
| FWD MMP9 | TTG ACA GCG ACA AGA AGT GG (SEQ ID NO: 11) |
| REV MMP9 | GCC ATT CAC GTC GTC CTT AT (SEQ ID NO: 12) |
| FWD Bcl2 | TTG TGG CCT TCT TTG AGT TCG GTG (SEQ ID NO: 13) |
| Rev Bcl2 | GTA CAG TTC CAC AAA GGC ATC CCA (SEQ ID NO: 14) |
| IL-6F RT | GGT ACA TCC TCG ACG GCA TCT (SEQ ID NO: 15) |
| IL-6R RT | GTG CCT CTT TGC TGC TTT CAC (SEQ ID NO: 16) |
| Fwd TERT | GAC TAC GTC GTG GGA GCC AG (SEQ ID NO: 17) |
| Rev TERT | CCT GTG GAT ATC GTC CAG GCC (SEQ ID NO: 18) |
| hTERC FWD | TCT AAC CCT AAC TGA GAA GGG CGT (SEQ ID NO: 19) |
| hTERC REV | TGC TCT AGA ATG AAC GGT GGA AGG (SEQ ID NO: 20) |
| STAT3 qPCR FWD | GAT CCA GTC CGT GGA ACC AT (SEQ ID NO: 21) |
| STAT3 qPCR REV | ATA GCC CAT GAT GAT TTC AGC AA (SEQ ID NO: 22) |

Mouse

| | |
|---|---|
| mBetaActin FW | CTG ACG GCC AGG TCA TCA CT (SEQ ID NO: 23) |
| mBetaActin RV | TAG TTT CAT GGA TGC CAC AGG AT (SEQ ID NO: 24) |
| mIKBa FW | GGC CAG CTG ACC CTG GAA (SEQ ID NO: 25) |
| mIKBa RV | GCC TCC AAA CAC ACA GTC ATC (SEQ ID NO: 26) |
| mIL6 FW | CAA AGC CAG AGT CCT TCA GAG A (SEQ ID NO: 27) |
| mIL6 RV | GCC ACT CCT TCT GTG ACT CCA (SEQ ID NO: 28) |
| mMCP1 FW | GCC CCT CCA TGT ATA CCA GAC T (SEQ ID NO: 29) |
| mMCP1 RV | AGA CCT CTC TCT TGA GCT TGG T (SEQ ID NO: 30) |
| mIL6 FW2 | GTT GTG CAA TGG CAA TTC TG (SEQ ID NO: 31) |
| mIL6 RV2 | CTC TGA AGG ACT CTG GCT TTG (SEQ ID NO: 32) |
| mIL1 FW | AAA GCT CTC CAC CTC AAT GG (SEQ ID NO: 33) |
| mIL1 RV | TCT TCT TTG GGT ATT GCT TGG (SEQ ID NO: 34) |
| mTNF FW2 | ACA GAA AGC ATG ATC CGC GAC (SEQ ID NO: 35) |
| mTNF RV2 | GAA GCC CCC CAT CTT TTG G (SEQ ID NO: 36) |
| mTERC FWD | TTT GTT CTC CGC CCG CTG TTT (SEQ ID NO: 37) |
| mTERC REV | AGC TCC TGC GCT GAC GTT TGT TT (SEQ ID NO: 38) |

### Chromatin Immunoprecipitation

ChIP was done from HeLa or A2780cp cells treated with TNFα for 45 or 60 min. Briefly, cells were fixed with 1% formaldehyde and whole cell lysates were sonicated to generate 200-500 bp fragments. Thereafter, the sonicated lysates was used for ChIP with anti-hTERT, anti-p65 or IgG control antibodies. After washing, the protein-DNA crosslinks were reversed and the DNA was eluted in 100 μL and was used for PCRs. GAPDH was used as negative control promoter. For ChIP-western, the IP was done as before, after washing, Laemelli loading dye was added directly to the beads, followed by Immunoblotting for bound proteins. For re-ChIP, first IP was done using anti-p65. After the washing, the ChIP eluate was used for IP again with anti-hTERT or anti IgG antibodies. The eluates from the reChIP were analyzed for binding NFκB dependent promoters by quantitative PCR. ChIP primers have been previously described¹³.

### ChIP DNA Library Construction and Sequencing

The quality of the ChIP DNA was controlled by checking the concentration and the enrichment of known targets in ChIP DNA over input with Quant-iT PicoGreen assay (Invitrogen) and real time PCR respectively. Libraries were then, constructed using SOLID ChIP-Seq Kit (Applied Biosystems) following manufacturer's protocol. For each sample, 10 ng of ChIP DNA was purified using AMPure XP Kit (Agencourt), end-repaired and ligated to SOLID adaptors. After ligation, samples were nick-translated and amplified using primers specific to adaptors for 15 cycles. Samples were purified multiple times between each step. Following the final purification step, size distribution and quantity of libraries were checked by performing DNA 1000 assay (Agilent). Samples with expected size distribution (165-365 bp) and quantity (min 2 ng/μl in 10 μl) were sequenced using SOLID4 sequencer following manufacturer's instructions for fragment sequencing. Each sample was loaded to a quarter SOLID slide.

### Sequencing Data Analysis

35 bp long reads (40-45×10̂6 reads per sample, 80% of all mappable reads per sample) were uniquely mapped to the Human Genome 19 (UCSC), using SOLID Bioscope 1.3.1 ChIP-Seq Module⁸⁹ For alignments, seed and extension approach was used. 30 bp seeds that aligned to the reference genome with at most 3 color mismatches were kept and extended up to 35 bp. Only reads with unique full length alignment (35 bp) allowing for 2 mismatches were used in the analysis. For each sample, data obtained from multiple spots were pooled together before further processing of the data.

Redundant reads (multiple reads aligning exactly to the same location), which are generally PCR-artifacts, were filtered. Control-based ChIP-seq Analysis Tool version 3 (CCAT3) was performed for peak calling under default settings using reads, which were mapped, to unique regions in the genome. Peaks with FDR superior than 0.2 are excluded from the analysis. For the downstream analysis, normalized fold enrichments reported by CCAT3 were used⁹⁰. Data was analyzed using UCSC genome browser⁹¹.

To assess the quality of peaks, CENTDIST motif enrichment tool was used under default settings⁹². For gene annotation of p65 peaks UCSC Ref-Seq annotation file was used⁸⁹. To plot the tag density profile, genomic region around each peak center (+/−5 Kb) was divided into bins of 100 bp, and the number of reads at each bin was counted. Then, read counts at bins with same distance relative to the peak center for all peaks were averaged. Finally, the average read count for each bin was normalized by the peak number and library sequence depth. For differential motif analysis, PWM of GGGTTAGGG motif was obtained from deNovo analysis of telomere repeat peaks using the SEME tool under the default settings⁹³. Then, the motif was scanned through TERT-dependent and TERT-independent peaks using the Motif Scan tool⁹².

Genomic Regions Enrichment of Annotations Tool (GREAT) was used to predict biological processes that p65 bound regions were involved in⁹⁴. Default settings were used.

### EMSA

Electrophoretic mobility shift assay (EMSA) was done as previously described¹³. NFκB consensus (double stranded, 5′-TCA ACA GAG GGG ACT TTC CGA GAG GCC-3′, (SEQ ID NO: 39)), ILEA (double stranded, 5′-ACT GGG AGG ATT CCC AAG GGG TCA ATT GGG AGA-3′, (SEQ ID NO: 40)), IL6B (double stranded, 5′-ACT GGG AGG ATT CCC AAG GGG TCAA-3′, (SEQ. ID NO: 41)), IL8 (IL8 A: 5′-TGA CTC AGG TTT GCC CTG AGG GGA TGG (SEQ ID NO: 42); IL8 B: 5′-TGA CTC AGG CCC GCC CTG AGG GGA TGG GCC-3′, (SEQ ID NO: 43)), TNFα (TNFα A, 5′-GCA TGG GAA TTT CCA ACT CTG GGA ATT CCA ATC CTT GCT GGG AA-3′, (SEQ ID NO: 44); TNFα B: 5′-GCA TGG GAA TTT CCA ACT CTC CCA ACC CCA ATC CTT GCT GGG AA-3′, (SEQ ID NO: 45)) and Oct-1 (double stranded, 5′-TGT CGA ATG CAA ATC ACT AGA A-3′, (SEQ ID NO: 46)) radiolabeled probes. Separation of the reaction was performed on a 6% non-denaturing polyacrylamide gel, which was then dried and analyzed with a PharosFX Plus system (BioRad, Hercules, Calif.). For supershift assays, 1 μg of IgG antibodies specific to members of NFκB proteins (Santa Cruz Biotechnology) and hTERT (Epitomics and Calbiochem) were added to nuclear extracts for 20 min on ice prior to addition of radiolabeled probes.

### Endotoxin Challenge and Kaplan-Meier Survival Curve

Wild-type mTerc^{-/-}, mTerc^{-/-}/Rap1^{+/-} or mTERT^{-/-} mutant age matched mouse cohorts (8-12 weeks old, n=6) were sensitized with GalN (700 μg/kg) for 20 min and then challenged with intraperitoneal injection of 50 μg/kg LPS (from Escherichia coli 0111:B4, Sigma L2630) in PBS, and survival monitored every hour for 24 hrs.

### Listeria Infection

Listeria monocytogenes was cultured in Brain-Heart Infusion (BHI; Difco 11059) broth. Mouse cohorts of indicated genotypes were injected intraperitoneally (I.P.) 10⁶ Listeria monocytogenes for bacterial challenge. After 0.3 days, mice were sacrificed and the spleens were harvested and the cells and other contents dissociated in 10 ml PBS. Serial dilutions of the splenic suspensions were plated on Brain-Heart Infusion (BHI; Difco 11065) agar plates and counted after 24-30 hrs. Bacterial clearance was measured as a function of number of bacterial colonies obtained from spleens of individual mice.

Culture of Primary Leukemia Cells from Patients

Bone marrow blast cells (>90%) from newly diagnosed leukemia patients were obtained at National University Hospital in Singapore. Primary leukemia cells were cultured in IMDM with 10% of fetal bovine serum (FBS), FLT3 ligand (20 ng/ml), SCF (20 ng/ml), IL-3 (20 ng/ml), G-CSF (50 ng/ml), TPO (50 ng/ml) and 1% Penicillin/Streptomycin in a humid incubator with 5% CO₂ at 37° C. All the human cytokines were purchased from Peprotech (Rocky Hill, N.J.). One day after in culture, one million leukemia cells each were seeded into 2 ml of medium with complete cytokines in a 6-well plated and were treated with MST-312 at 0.5 μM or 0.1% dimethyl sulfoxide (DMSO) as control for 48 hours before harvested for RNA extraction.

### Results

### Functional Intersection Between Telomerase and NFκB Signalling

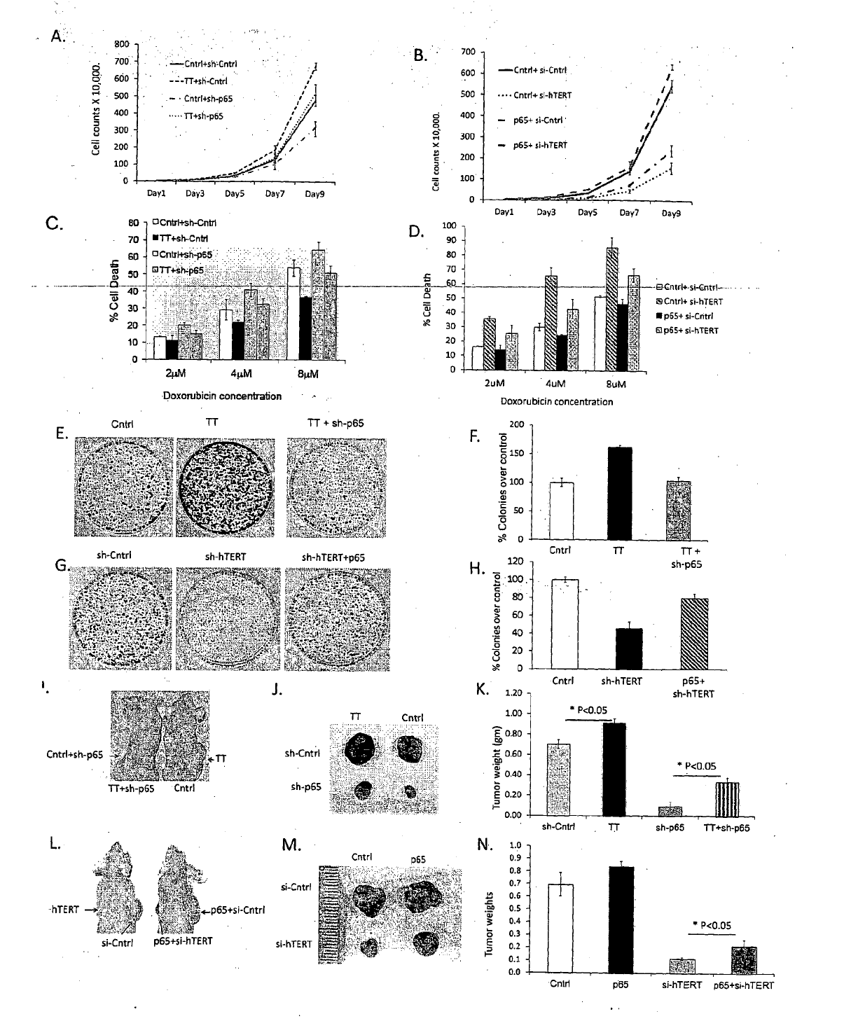

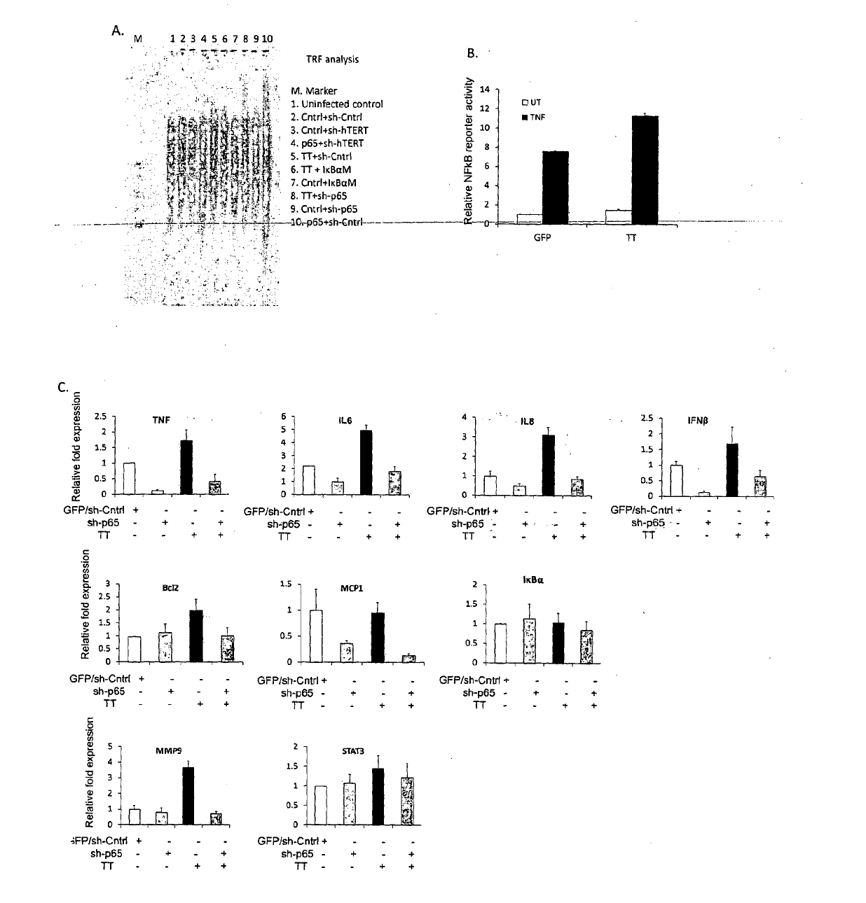

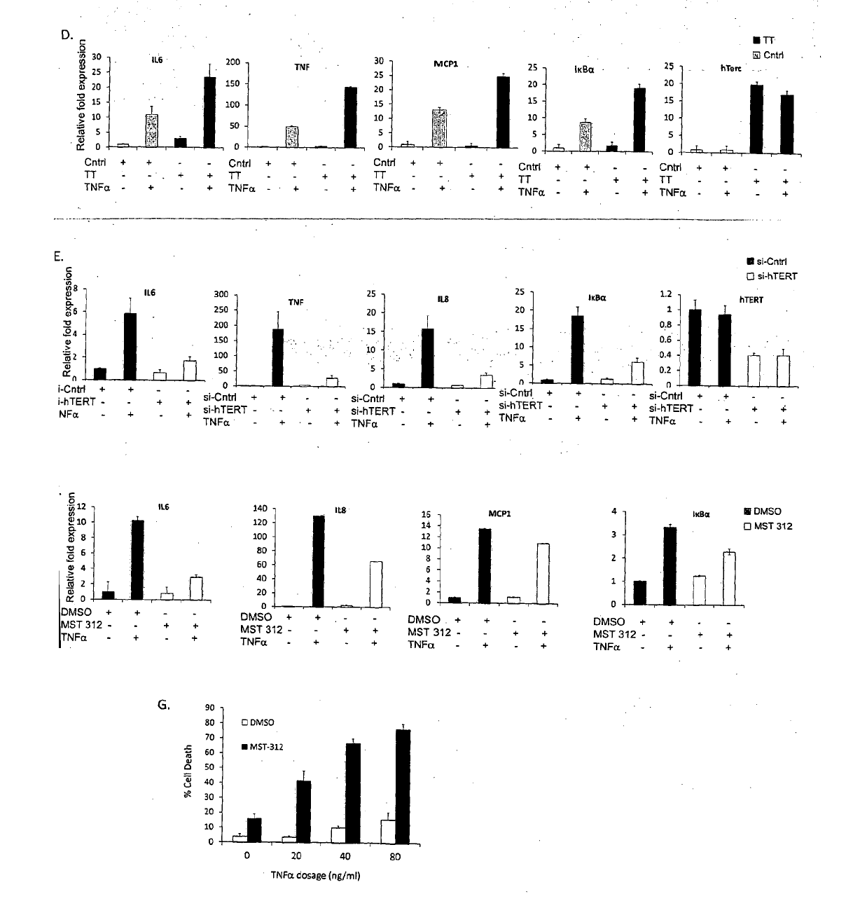

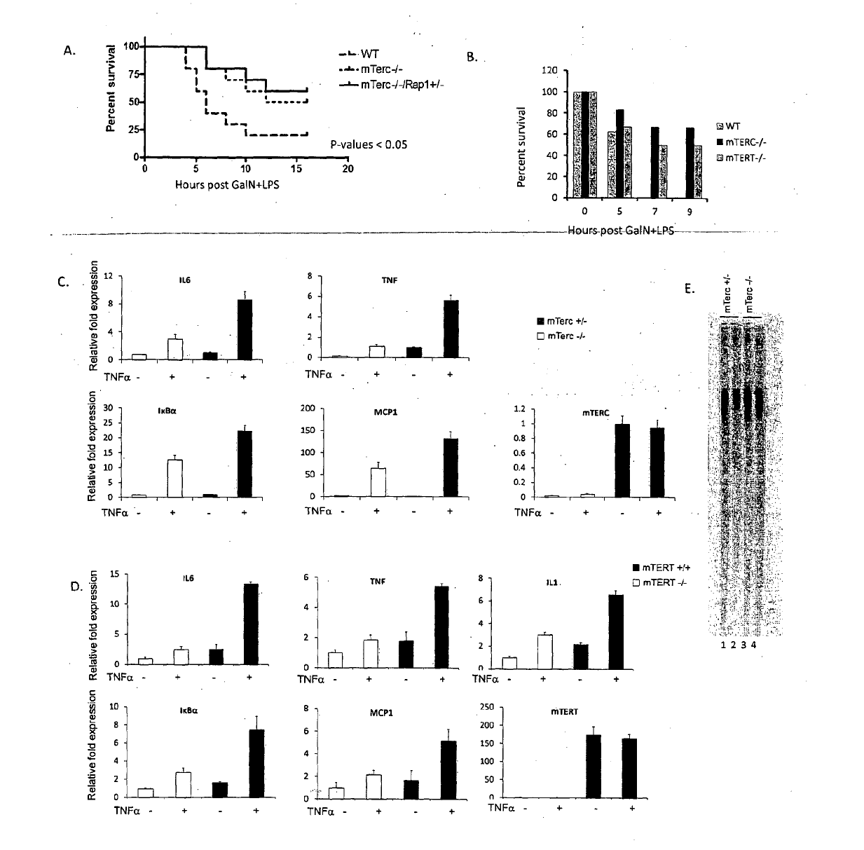

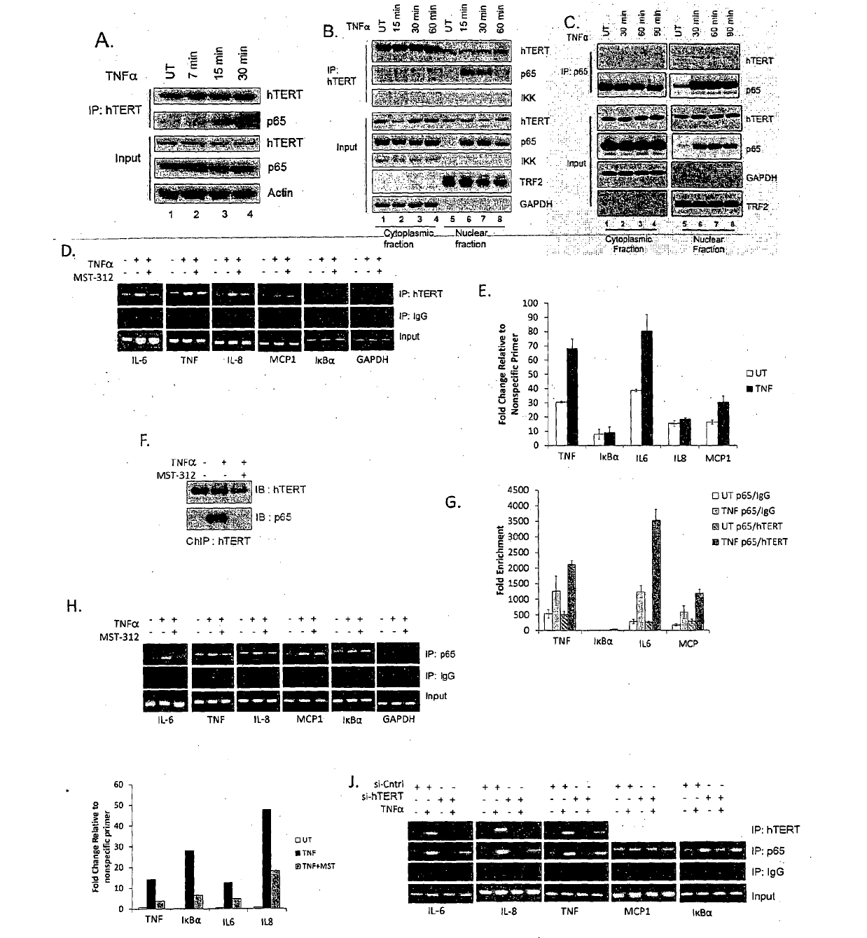

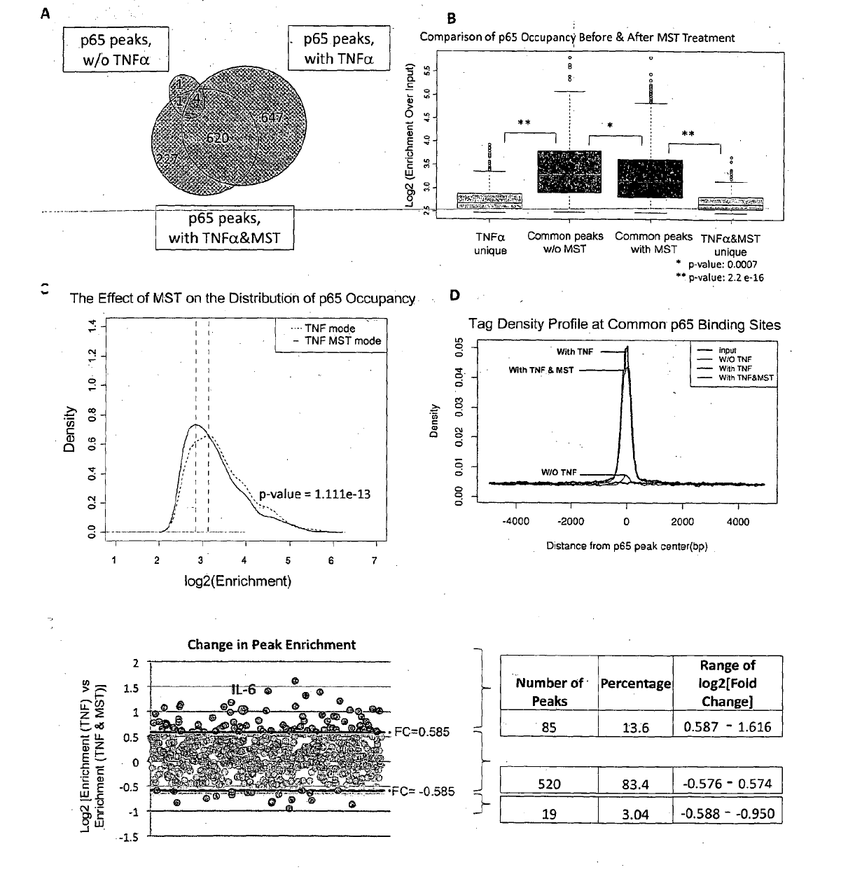

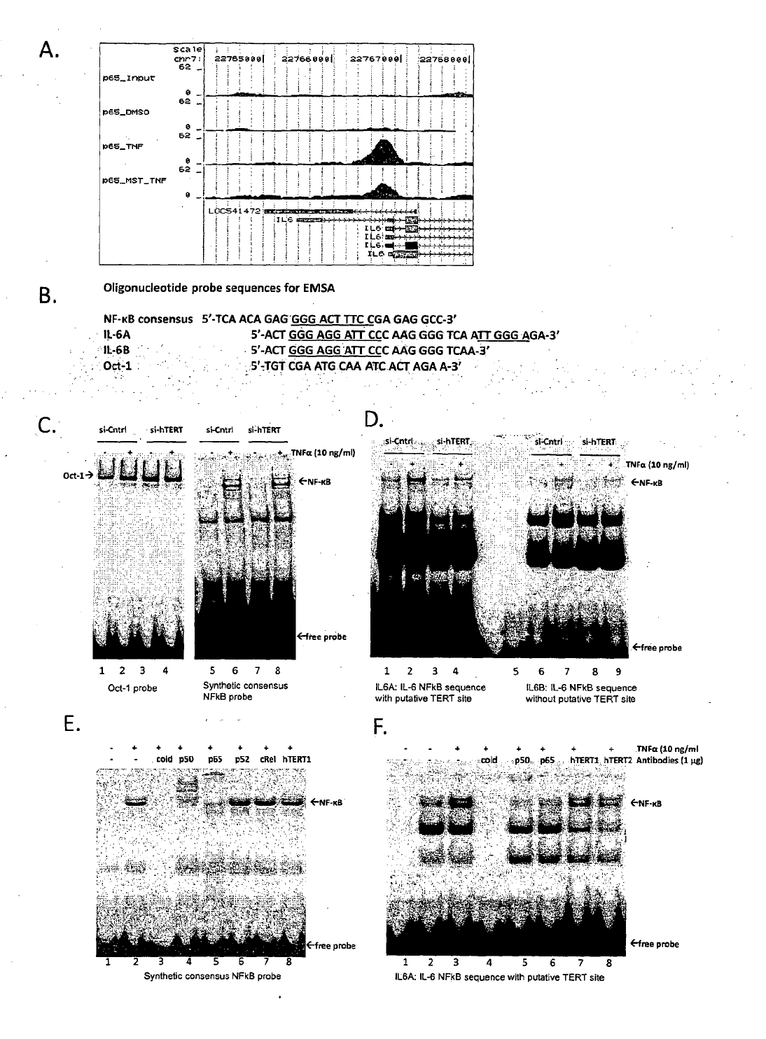

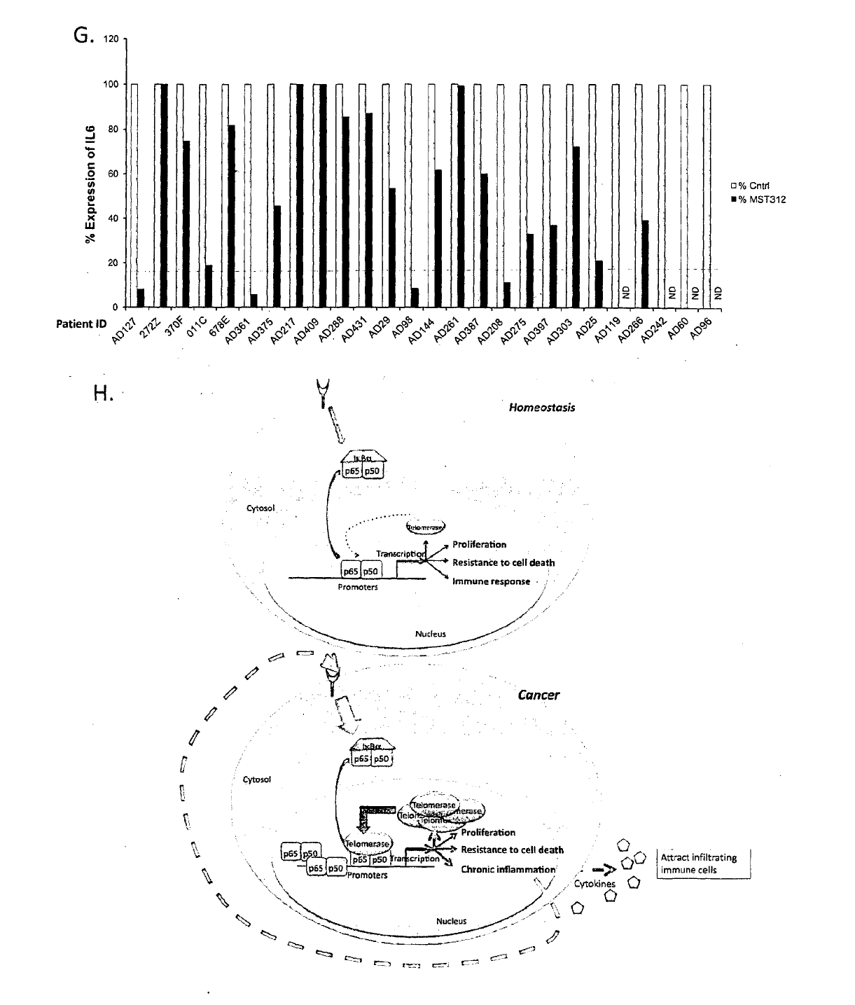

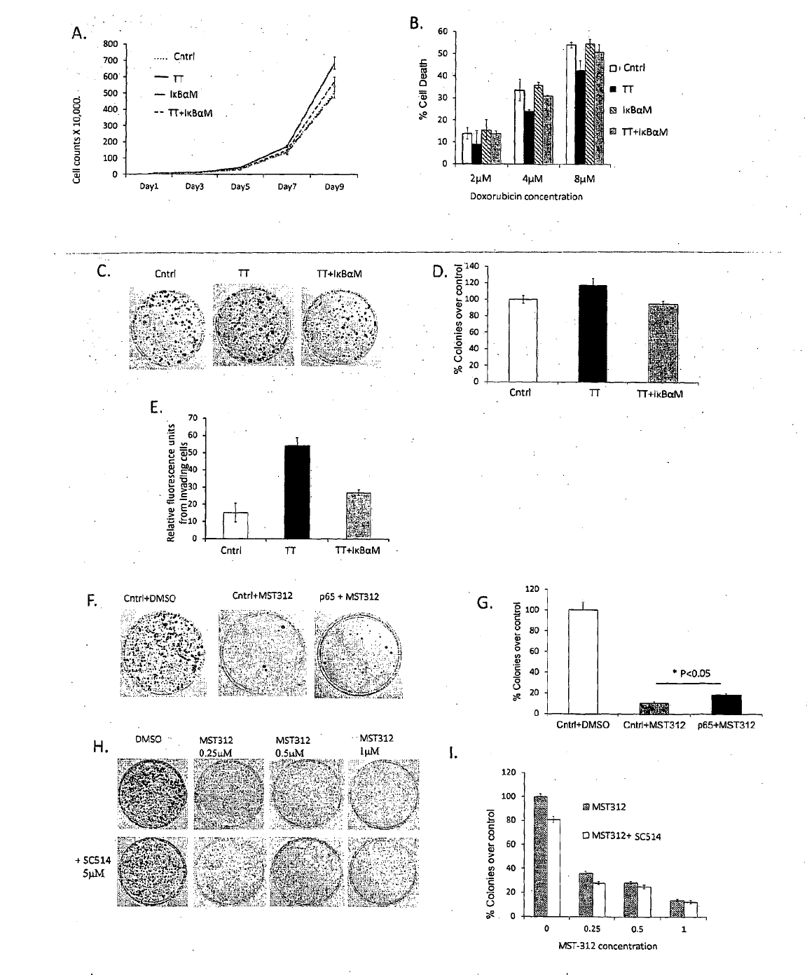

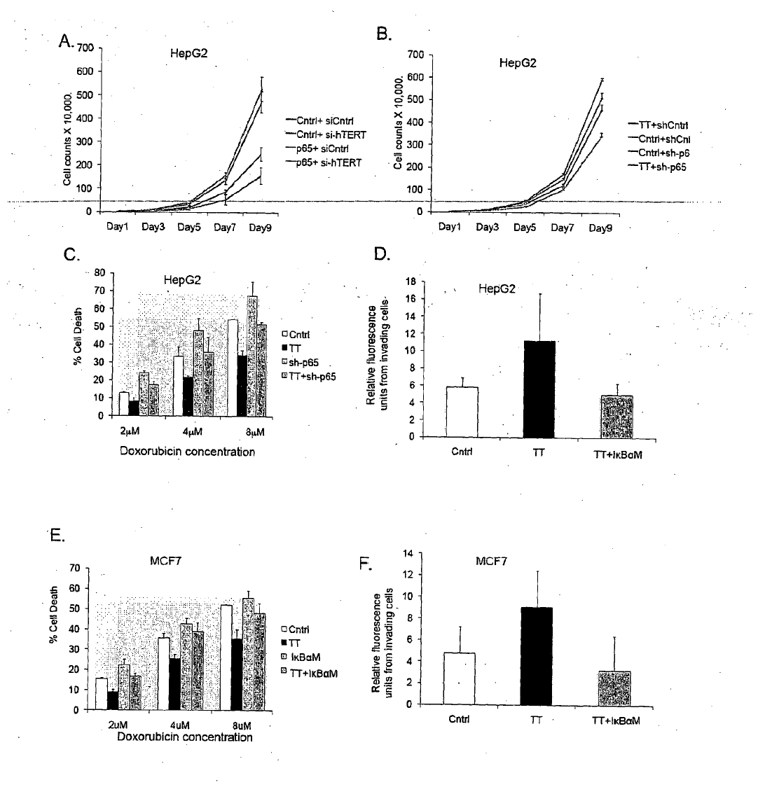

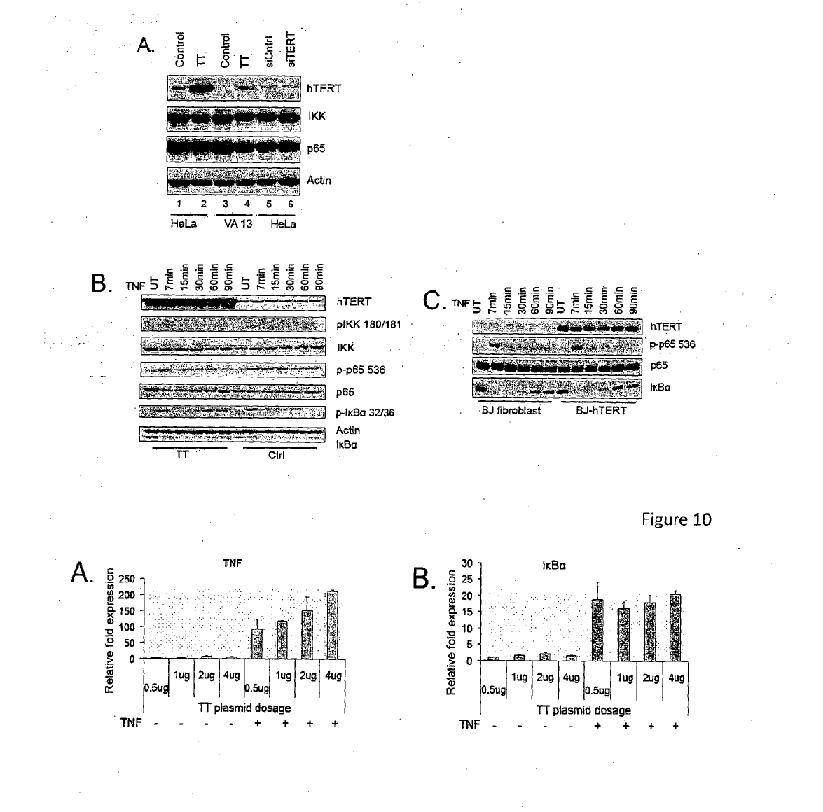

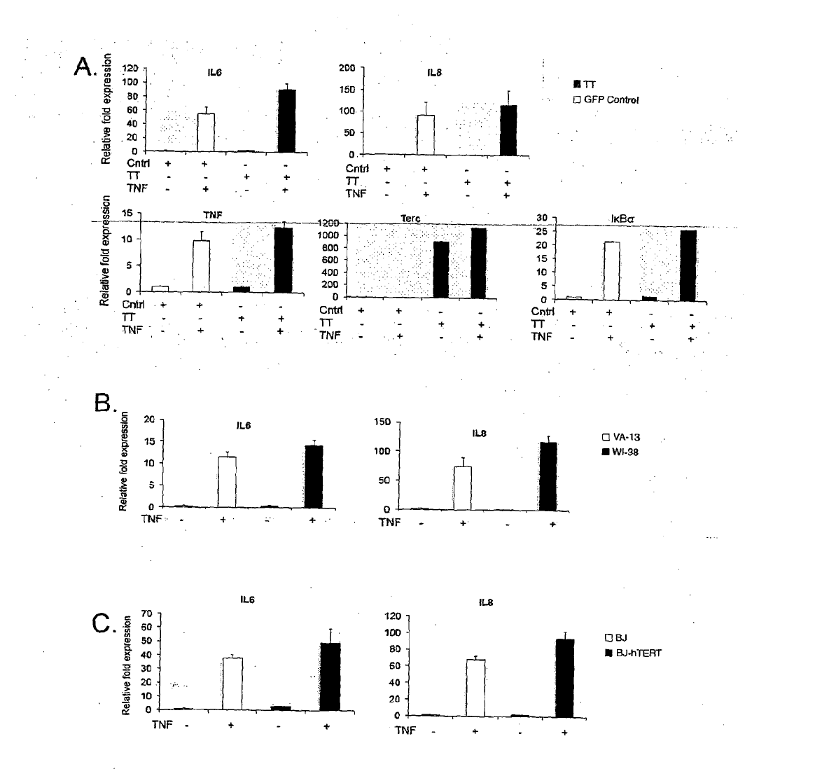

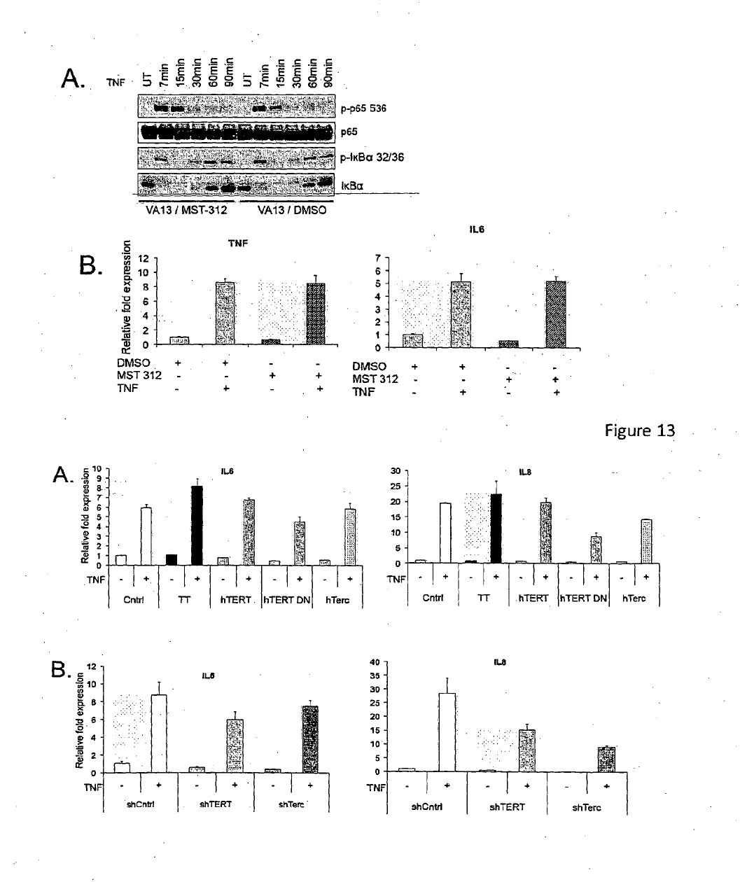

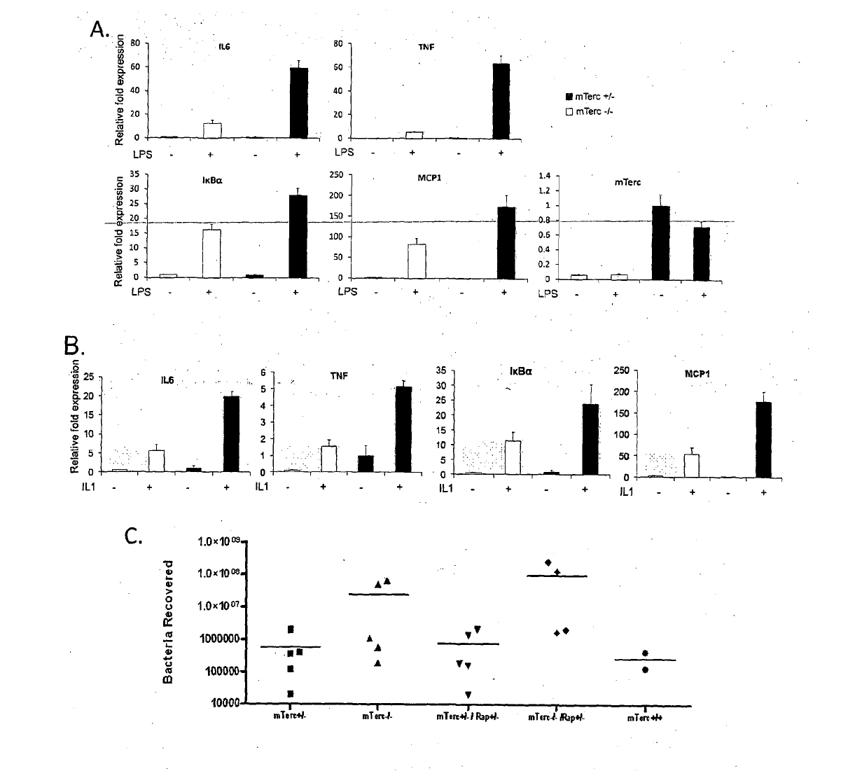

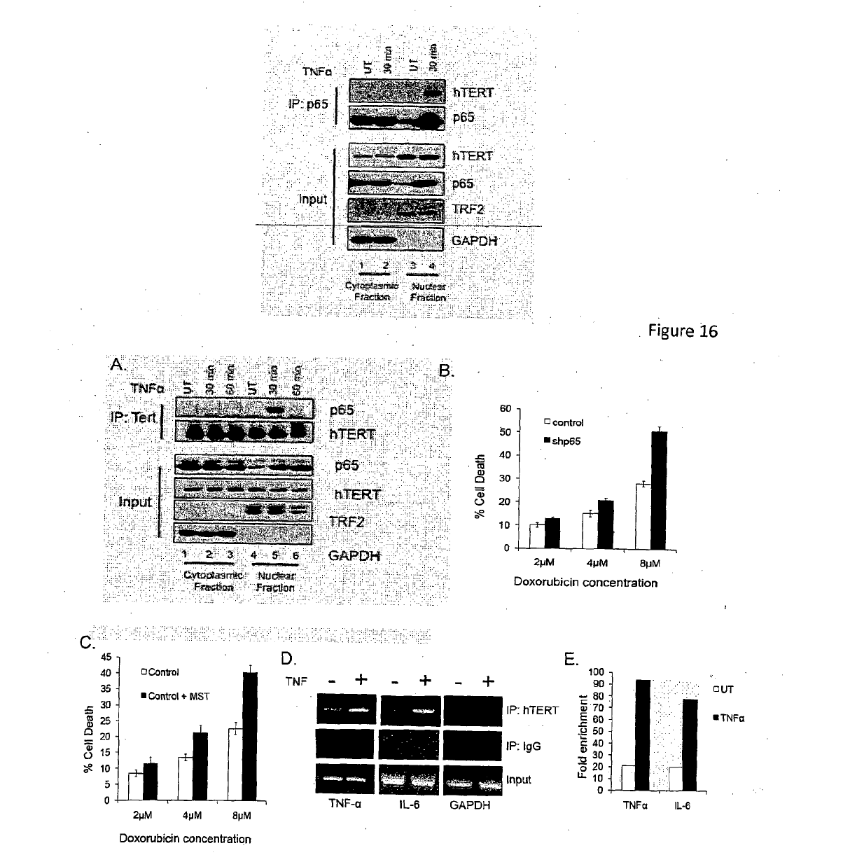

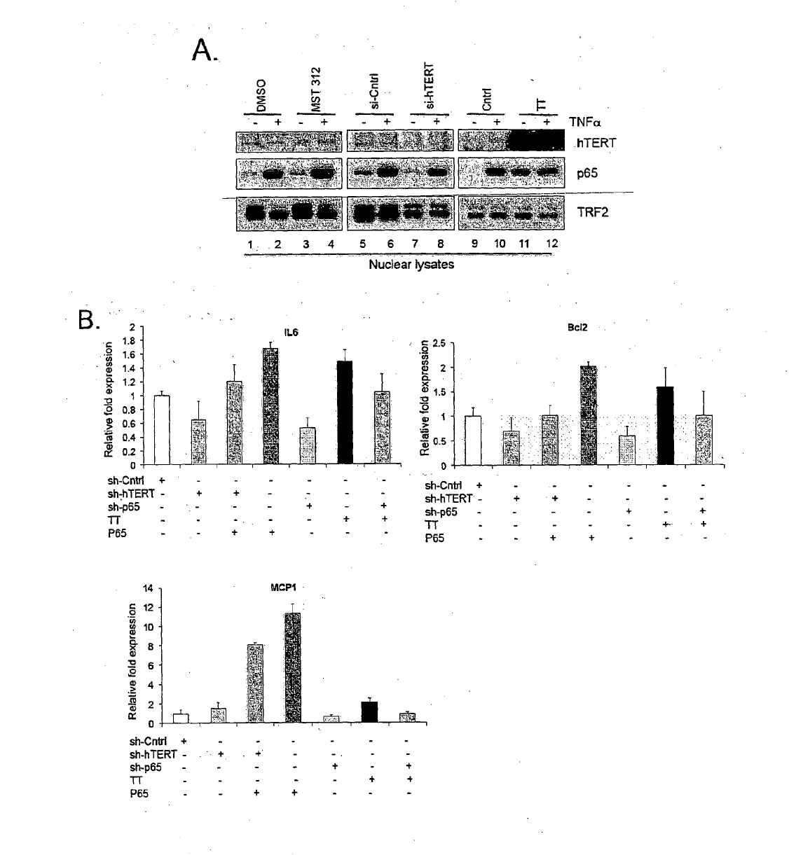

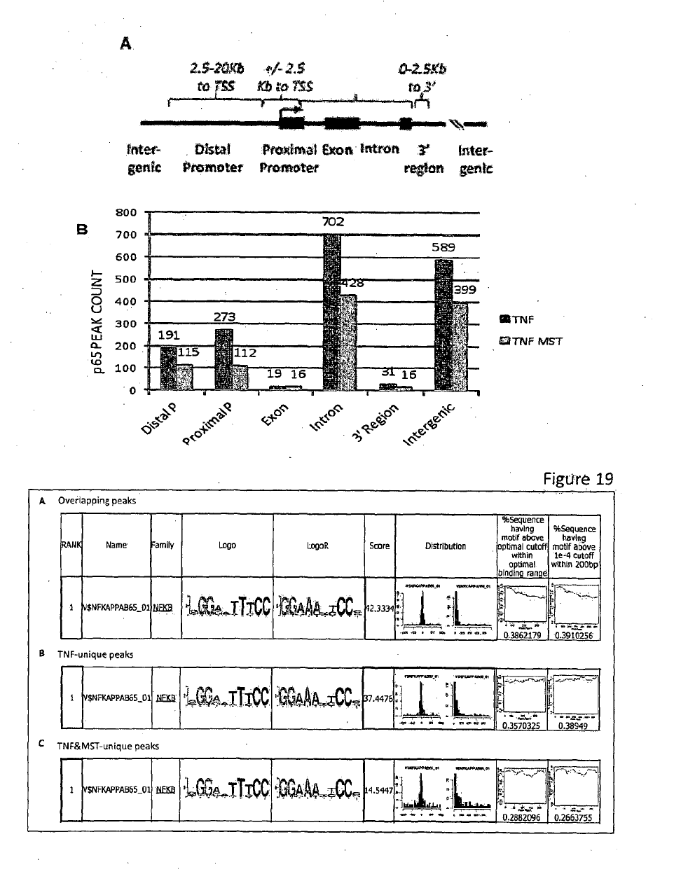

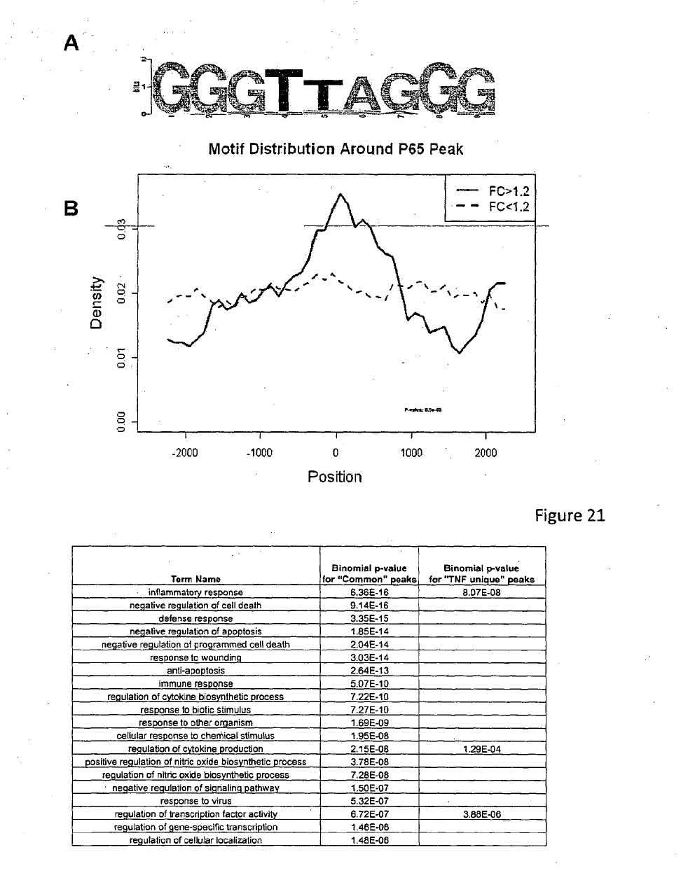

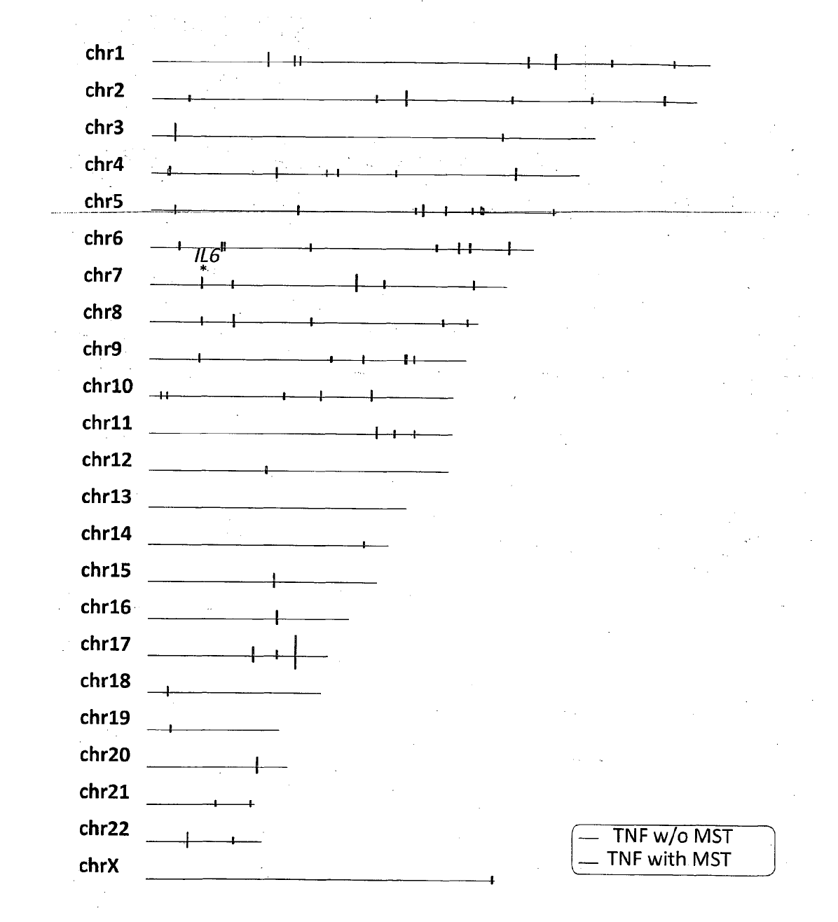

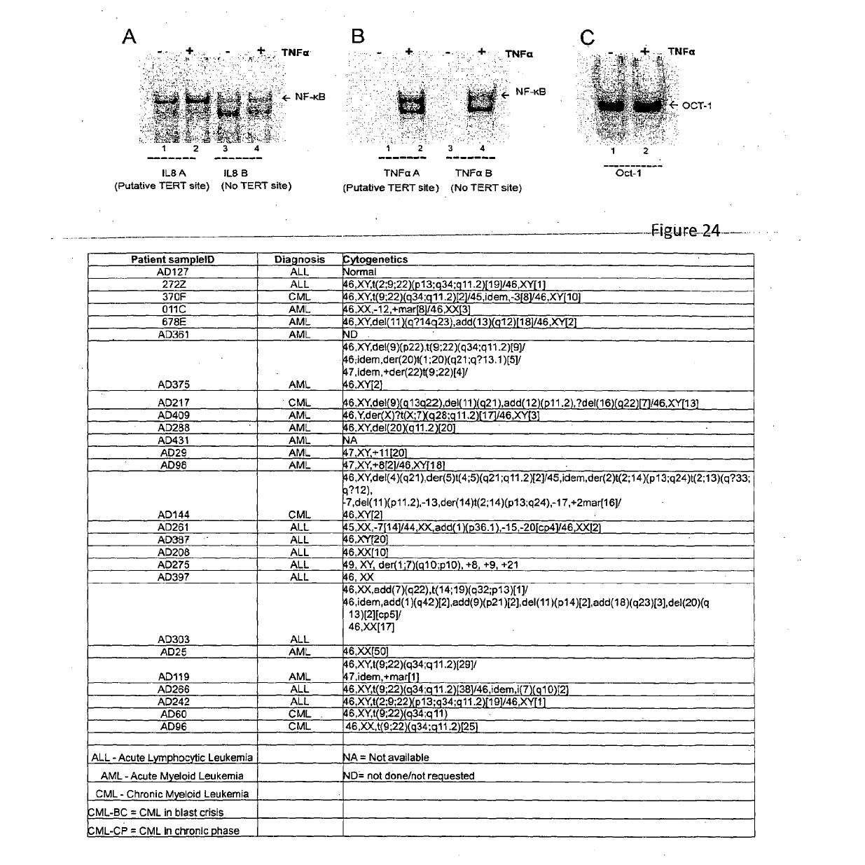

To investigate if telomerase and NFκB signalling functionally intersect, telomerase components hTerc+hTERT (TT) were ectopically expressed with or without concurrent inhibition of NFκB achieved either by shRNA to NFκB p65 subunit (sh-p65) or by overexpression of a transdominant IκBαM mutant (FIG. 7)⁶⁶. While ectopic expression of telomerase led to increased cell proliferation, the increased proliferative potential of these cells could be reduced to baseline levels when NFκB signalling was simultaneously dampened by sh-p65 (FIG. 1A). Conversely, reduction of telomerase levels by siRNAs to hTERT (si-hTERT) led to reduced cell proliferation which could be partially but reproducibly rescued by ectopic expression of p65 (p65) (FIG. 1B). Ectopic expression of telomerase protected cells from death induced by doxorubicin, but simultaneous inhibition of NFκB nullified the protective effect of telomerase on cell survival (FIG. 1C). Conversely, while si-hTERT cells were more sensitive to chemotherapy induced cell death, these cells could be protected by ectopic p65 expression (FIG. 1D). While ectopic expression of telomerase increased the number and size of colonies, sh-p65 significantly reduced the number of colonies formed under these conditions (FIGS. 1E & F). Notably, reduction in colony forming ability of cells due to hTERT ablation (sh-hTERT) could be significantly rescued by ectopic expression of p65 (FIGS. 1G & H). Much like sh-p65 expression, inhibition of NFκB signalling via expression of IκBαM mutant protein also had similar effects on cell proliferation (FIG. 7A), cell death (FIG. 7B) and colony formation (FIG. 7C-D). Furthermore, inhibition of NFκB signalling via IκBαM mutant also blocked increased invasive capacity afforded to cells by telomerase expression (FIG. 7E). These data were replicated in other primary (FIG. 16 B-C) and cancer cells (FIG. 8).

The inventors next sought to test another means of reducing functional telomerase levels and employed MST 312⁶⁷, a previously described chemical inhibitor, of telomerase activity. Indeed, chemical inhibition of telomerase activity phenocopied the results of hTERT knockdown which were partially rescued by ectopic expression of p65 (FIG. 7F-G). While SC514, an IKK inhibitor reduced the number of colonies formed, the effects were not significant when used in combination with MST312 (FIG. 7H-I).

FIGS. 1I-K show that ectopic expression of telomerase led to larger tumors in a xenograft model, but blocking NFκB reduced tumor size and weight (p<0.05 by two tailed student t-test). Furthermore, ectopic expression of p65 significantly restored the ability of si-hTERT cells to form tumors in the xenograft model (FIG. 1L-N). Taken together, it was concluded that expression of telomerase as seen in cancer cells could have many consequences, which include increased protection against cell death, increased proliferation, increased colony forming ability and increased invasion, and that at least a part of these effects require the simultaneous functioning of NFκB signalling.

### Telomerase Regulates NFκB Dependent Gene Expression

Telomere restriction fragment length analysis showed that telomere lengths in cells where the levels of telomerase components or NFκB signalling components were manipulated did not vary significantly (FIG. 2A). The inventors next addressed if telomerase can directly regulate NFκB signalling to mediate some of the effects seen in the assays. Ectopic expression of telomerase led to increase in NFκB dependent reporter (FIG. 2B) and endogenous genes (FIG. 2D) in response to TNFα, a known stimulator of NFκB signalling. Ectopic expression of telomerase led to significant increase in expression of a number of endogenous NFκB targets even without any stimulation which could be negated by sh-p65 (FIG. 2C). Telomerase mediated regulation of NFκB dependent gene expression displayed selectivity wherein some NFκB targets such as MCP1 and IκBα were not significantly affected merely by telomerase expression (FIG. 2C).

Specificity of the hTERT antibody was rigourously tested (FIG. 9A). Telomerase failed to induce changes in the activation of IKKs, degradation of IκB or phosphorylation of p65 at serine-536 (FIG. 9B-C) when cells were challenged with TNFα suggesting that telomerase mediated regulation of NFκB pathway occurs downstream of IKK activation and p65 phosphorylation. Telomerase (TT) could activate endogenous NFκB target genes in dose (FIGS. 10A&B) and stimulus (FIG. 11A) dependent manner in telomerase proficient (293T) and telomerase null (VA13) cells. Furthermore, the inventors compared gene expression between paired sets of cell lines that have inherently different levels of telomerase expression (FIG. 11B). The VA13 telomerase null cells were compared to Wi38 telomerase WT cells and BJ telomerase null normal fibroblast was compared to BJ-hTERT (telomerase reconstituted) cells (FIG. 11B-C). Both VA13 and BJ cells reproducibly had significantly lesser levels of IL6 and IL8 activation compared to Wi38 and BJ-hTERT cells (FIG. 11B-C).

TNFα induced expression of NFκB targets was reduced when cells were treated with siTERT (FIG. 2E) or MST-312 (FIG. 2F). In these experiments, it was observed that the effect of loss of telomerase is more pronounced on expression of genes like IL6 or TNF as compared to MCP1 and IκBα reiterating the differential regulation (FIG. 2E-F). A key function of NFκB is to protect cells from TNFα induced apoptosis⁵⁶. Indeed, MST-312 treatment sensitized cells to TNFα induced cell death (FIG. 2G).

To confirm that MST-312 specifically inhibits telomerase without affecting NFκB signalling directly, VA13 cells were treated with MST-312 or DMSO and stimulated with TNFα in a time dependent manner (FIG. 12A). No changes were observed in p65 phosphorylation or IκBα degradation upon MST-312 treatment. MST-312 treatment also did not affect stimulus dependent NFκB gene expression in VA13 cells (FIG. 12B). These data indicate that at the dosages used, MST-312 does not directly interfere with NFκB signalling. Furthermore, short term MST-312 treatment did not cause changes in levels of hTERT (FIG. 12B). Telomerase holoenzyme enhanced gene expression much better than hTERT or hTerc alone (FIG. 13A-B). Taken together with data in FIG. 2D-F, these results suggest that telomerase holoenzyme is important for directly regulating NFκB dependent gene expression.

### Telomerase Null Mice Display Defective NFκB Signalling

To examine if telomerase indeed regulates NFκB signalling in vivo, the inventors evaluated if loss of functional telomerase has a bearing on the ability of animals to mount a response to endotoxins, a function well know to be orchestrated by NFκB signalling⁶⁸. First generation telomerase null (mTerc^{-/-}) and littermate control mice were treated with LPS after sensitization with GalN⁶⁹. Survival of animals was monitored hourly post injections. Kaplan-Meir plot of the experiment suggested that mice lacking functional telomerase (mTerc^{-/-}) are resistant to endotoxic shock with more than 50% surviving at the end of the experiment in contrast to control littermates (FIG. 3A). The mTerc^{-/-} mice were crossed to Rap1 mutant mice¹³ which are endotoxin resistant¹³. mTerc^{-/-}/Rap1^{+/-} mice were more resistant to endotoxic shock compared to the mTerc^{-/-} alone or Rap1^{+/-} alone (data not shown) groups (FIG. 3A). Much like the mTerc^{-/-} mice, mTERT^{-/-} mice were also resistant to endotoxic shock compared with wild-type controls (FIG. 3B).

To evaluate the molecular reason for the endotoxin resistance of telomerase null mice, multiple independent pairs of mTerc^{-/-} and mTERT^{-/-} MEFs and their corresponding control primary embryonic fibroblasts were established. Indeed, compared to control MEFs, mTerc^{-/-} (FIG. 3C) and mTert^{-/-} MEFs (FIG. 3D) were defective in activating NFκB dependent gene expression upon stimulation with TNFα or other NFκB activating stimuli such as IL1 and LPS (FIG. 14A-B). Similar results were obtained in all the pairs of MEFs examined (data not shown). Since these MEFs were established from 1^{st} generation mTerc^{+/-} matings, the mean telomere lengths of mTerc^{-/-} and mTerc^{-/-} cells were comparable suggesting that the effects of telomerase on NFκB dependent inflammatory gene expression are independent of telomere dynamics (FIG. 3E). mTerc^{-/-} mice are also less successful in clearing Listeria monocytogenes⁶⁸ when compared to the mTerc^{+/-} littermates (FIG. 14C). Based on these evidences, it was concluded that functional telomerase is a regulator of NFκB dependent inflammatory program in vivo.

### Telomerase Binds to p65 and Localises to a Subset of NFκB Promoters

Modulation of telomerase levels did not seem to affect the cytosolic signalling arm of the NFκB cascade. Hence, the inventors tested whether telomerase associates with NFκB in the nucleus. Co-immunoprecipitation experiments showed that hTERT associates with p65 within 15-30 minutes post stimulation with TNFα (FIG. 4A) mainly in the nuclear fraction of transformed (FIGS. 4B&C) and primary lines (FIGS. 15 & 16). Although there remained a substantial amount of both p65 and hTERT in the cytosol at all time points, the inventors did not find a strong association between them indicating that the nuclear translocated pool of p65, that was free of IκBα, was the pool that telomerase can associate with. These data suggest that telomerase mediated regulation of NFκB occurs in the nucleus, probably at the level of DNA binding. Therefore, chromatin immunoprecipitation from cells treated with either DMSO (as control) or MST-312 prior to TNFα treatment was performed. It was observed that telomerase binds to promoters of IL6, TNF and IL8 robustly upon stimulation (FIG. 4D & FIG. 16D) and this binding was reduced upon MST-312 treatment (FIG. 4D). Recruitment to MCP1 promoter was comparatively weak and almost did not occur on the IκBα promoter (FIG. 4D-E). These differences in binding may be the reason for the differential effects of telomerase mediated modulation of NFκB targets in human (FIG. 2) and murine cells (FIG. 3). Overexpression of telomerase led to nuclear stabilisation of p65 (FIG. 17A) even without stimulation. However, simply inhibiting telomerase activity by MST-312 or by si-hTERT did not affect TNFα stimulated nuclear translocation of p65 (FIG. 17A). Since telomerase overexpression can increase nuclear residence/stability of p65, it explains why ectopic expression of telomerase is sufficient to activate NFκB target genes (FIG. 2C, 17B).

A ChIP-western was next performed, and it was found that p65 associates with hTERT on chromatin upon TNFα stimulation in a MST-312 sensitive manner (FIG. 4F). By performing re-Chip; a ChIP of hTERT from the eluate of p65 ChIP after TNFα stimulation, it was verified that these two proteins associate with differential strengths on distinct NFκB promoters (FIG. 4G). p65 binding was also strongly inhibited by MST-312 treatment (FIG. 41). Strikingly, TNFα induced binding of p65 to IL6, TNF or IL8 promoters but not IκBα promoter was significantly reduced in si-hTERT cells (FIG. 4J). Taken together, this data suggests a mechanism where telomerase is recruited to select NFκB dependent promoters upon stimulation. This recruitment occurs concurrently with p65 and also mirrored by its association with p65 upon stimulation. Furthermore telomerase is required for optimal binding of p65 to a subset of NFκB dependent promoters.

### Telomerase Inhibition Attenuates Genome-Wide TNFα -Dependent p65 Binding on a Fraction of Target Sites

Chromatin immunoprecipitation sequencing (ChIP-seq) was carried out to analyze the effect of telomerase inhibition on TNFα-induced genome-wide p65 binding. It was observed that TNFα treatment increased p65 occupancy at 1271 regions, which were mostly intergenic and intronic sites (FIG. 18). MST-312 treatment prior to TNFα stimulation, reduced the number of p65 binding sites to 852 out of which 228 were new binding sites (FIG. 5A). The peaks that overlap in presence and absence of MST-312 were referred to as the “common” peaks, and peaks, which were completely lost or gained due to MST-312 treatment were referred to as “TNF-unique” and “TNF&MST-unique” peaks, respectively. Motif analysis on each subset of peaks showed a strong NFκB motif enrichment with a good score and a distribution centered around peak summit (FIG. 19). A motif analysis showed significant enrichment of telomeric repeats in proximity to MST-312 sensitive p65 peaks (FIG. 20). However, it was observed that p65 binding at the common peaks were significantly stronger than the unique peaks (FIG. 5B). The weak binding at unique peaks suggests that they are less likely to be stable/important binding sites. To further explore this premise, the inventors identified the top 20 biological processes that each group of p65 binding sites might be involved in by using Genomic Regions Enrichment of Annotations Tool (GREAT). GREAT predicted that the genes associated with common peaks were correlated with several functions related to inflammation and anti-apoptosis (FIG. 21). While TNF unique binding sites had a similar number of peaks as the common set, only a few of the functional categories were associated with these unique peak sets (only 3 out of the 20 processes). There was no significant biological process associated with TNF&MST-unique peaks. This suggested that the common peaks engage the more important genes in NFκB function whereas genes associated with TNF&MST-unique binding sites, in particular are likely to have no relevance to the p65-TERT interaction. Therefore, the inventors focused their analyses on p65 targets in the common peaks. When the effect of MST-312 on binding strength of p65 at the common sites was evaluated, the distribution of peak enrichment over input DNA significantly shifted to a lower intensity profile (FIG. 5C) when cells were treated with MST-312 prior to TNFα. This suggests a decreased binding of p65 in the presence of MST-312 (FIG. 5C). On detailed analysis, this reduction of p65 occupancy by MST-312 was evidently restricted to a subset of binding sites (FIG. 5D)⁷⁰. Only 13% of the common peaks showed a reduced occupancy with a minimum fold change of 1.5, after MST-312 exposure (FIGS. 5E, 22). Taken together, the data indicates that telomerase is indeed required for optimal p65 binding but on a small proportion of NFκB target sites upon inflammatory stimuli.

### Telomerase Regulates p65 Binding on Specific NFκB Target Gene Promoters

Genome-wide ChIP-seq analysis showed that p65 peak at the promoter of IL-6 was among regions where binding was affected most by MST-312 with a fold change of 2 (FIGS. 5E, 6A). To further clarify the mechanism by which telomerase regulates p65 binding on certain target genes, electrophoretic mobility shift (EMSA) and supershift assays using consensus NFκB sequence and NFκB sequences from IL6 promoter were carried out (FIG. 6B). While TNFα stimulation increased NFκB binding, levels of hTERT did not affect NFκB binding under basal or stimulated conditions (FIG. 6C). However, when an oligo derived from IL6 promoter (IL6 A) spanning the NFκB site was used as probe (FIG. 6B), hTERT ablation significantly reduced TNFα stimulated NFκB binding (FIG. 6D; compare lanes 2 to 4). Apart from the NFκB binding site within the IL6 A, this 33-mer oligo also contained a T₂G₃ sequence (which could be a putative hTERT binding site) in proximity. To directly evaluate the contribution of the T₂G₃ sequence in NFκB binding, the inventors used the IL6B oligo which lacked the T₂G₃ sequence but retained the NFκB binding site. Indeed, IL6 promoter without the T₂G₃ sequence showed significantly dampened NFκB binding (FIG. 6D; compare lane 2 to 6). The reduction in binding was comparable to that seen on IL6A oligo when lysates from si-hTERT cells were used (FIG. 6C; compare lane 4 to 6). These data indicate that telomerase mediated regulation of p65 binding is specifically dependent on sequence context and possibly dependent on the presence of a T₂G₃ sequence.

The inventors next evaluated if hTERT is physically present on the IL6 promoter by performing a supershift assay (FIGS. 6E and F). While antibodies to the p50 and p65 of NFκB could supershift complexes on the consensus NFκB oligo (FIG. 6E; compare lanes 2 and 3/4), antibodies to hTERT, p52 and c-Rel (NFκB subunits as controls) did not supershift these complexes (FIG. 6F; compare lanes 2 and 8). However, when the same experiment was repeated using the IL6A oligo which contains the putative hTERT binding T₂G₃ sequence, two independent hTERT antibodies significantly disrupted the NFκB complexes (FIG. 6E; compare lanes 3 and 7/8). As expected, both p50 and p65 antibodies supershift these complexes verifying their validity as NFκB complexes (FIG. 6E; compare lanes 3 and 5/6). These observations were further validated on two independent NFκB promoters, IL8 and TNF (FIG. 23). These data provide direct evidence that telomerase binds to certain NFκB promoters like IL6 promoter and that it has a direct role in regulating the binding of NFκB per se. These results also explain how telomerase could be a direct regulator of NFκB dependent genes which include inflammatory cytokines and genes associated with transformation.

### Telomerase Inhibition Reduces IL6 Expression in Primary Human Cancers

The inventors finally sought to establish if the mechanism of telomerase mediated transcriptional regulation of NFκB targets is also seen in primary cells derived from cancer patients. Primary leukemic cells from AML, ALL or CML patients were obtained from the National University Hospital, Singapore (FIG. 24). Gene expression analysis from these primary cancers showed that in more than 80% of the samples, the levels of IL6, a representative NFκB target gene were significantly reduced by MST-312 treatment (FIG. 6G). These results reiterate that inhibiting telomerase activity in cancers could be an effective means of blocking NFκB target genes which aid in inflammation/transformation.

### DISCUSSION

While synthesizing telomeric DNA is a well recognized function of telomerase, recent evidence suggests that this enzyme plays roles in other biological processes^{15,22}. However, the mechanism by which telomerase contributes to these processes which are critical for transformation is not very clear. In this study, the inventors uncovered that telomerase can directly regulate NFκB dependent transcription. Furthermore, the inventors demonstrated that NFκB signalling functionally contributes to telomerase function in processes relevant to transformation (FIG. 1). Given that NFκB is hyperactivated in a equally large number of cancers as telomerase, and that NFκB is well documented to positively regulate several genes important for cell proliferation, resistance to apoptosis and invasion, the results presented herein might have uncovered a key missing molecular link that mediates effects of reactivated telomerase in cancer cells.

It is well known that NFκB regulates hTERT expression by binding to a site 350 bases upstream of the translational start site^{71,72,73}. In this study, the inventors made the striking observation that a number of telomerase dependent functions rely, at least in part, on its ability to activate NFκB in turn. This function of telomerase is reliant on its ability to directly bind a subset of NFκB target genes (FIG. 4) and turn on NFκB dependent transcription (FIG. 2). The telomerase holoenzyme is the most proficient (compared to catalytic subunit by itself) in regulating NFκB dependent genes and it mediates this activity in the nucleus by directly binding DNA (FIG. 4). Using a range of biochemical assays, it was shown that telomerase mediated NFκB target gene expression is regulated by physiologically relevant NFκB activating stimuli and this activation obeys the kinetics followed by other known regulators of the pathway (FIG. 4B-C).

The inventors also showed that mice lacking functional telomerase are defective in mounting an immune response upon LPS challenge, a function dependent on efficient NFκB signalling (FIG. 3). A recent report⁷⁴ shows that compared to age matched disease-free controls, the circulating PBMCs in patients with metabolic syndromes (MS), where inflammation also serves as a driver of pathology, produce enhanced levels of TNFα and IL6 and have high levels of telomerase activity. While this study⁷⁴ did not suggest any mechanism, the requirement of telomerase in maintaining telomere length during clonal expansion processes was assumed to be the underlying mechanism for the observations. The inventors suggest that telomerase mediated modulation of NFκB dependent cytokines which are key for development and function of a number of hematopoietic cells could be a major reason for the inflammatory gene-expression in MS patients⁷⁴ and for immune deficiencies observed in telomerase null cells/mice^{39,75,76}. A recent, report shows that telomerase mutant mice are very susceptible to ulcerative typhlocolitis associated with Helicobacter mastomyrinus⁷⁷, thereby providing an independent validation of the inventors' data from the bacterial infection model (FIG. 13C). Although the inventors' results unequivocally show that telomerase is a important regulator of NFκB signalling in vivo, one obvious difference between the mTerc^{-/-} or mTERT^{-/-} mice and NFκB p65 deficient mice, is that the telomerase null mice do not die embryonically like the p65 deficient mice⁷⁸. These differences could be explained by the fact that telomerase functions as a modulator of NFκB activity and that not all NFκB target genes are regulated by telomerase (FIG. 5).

Transcriptional regulation by telomerase has been previously reported in the context of wnt signalling¹⁰. Although telomerase mediated regulation of wnt target genes depends on the ability of telomerase to recruit a chromatin regulator Brg-1¹⁰, the inventors did not observe any change in association of hTERT with Brg-1 upon stimulation with TNFα (data not shown). Park et al. also observed that a catalytically incompetent, dominant-negative mTERT protein was able to rescue the wnt defect in mTERT^{-/-} mice suggesting that the RNA component Terc was not required in this context¹⁰. However, in the context of NFκB signalling, the inventors observe that the telomerase holoenzyme, in human cells, is most proficient upregulating target gene expression compared to either wild-type hTERT or the dominant-negative hTERT. Inhibition of telomerase activity by MST-312 phenocopied the gene expression and ChIP results from si-hTERT experiments. Although the exact mode of action of telomerase inhibition by MST-312 remains unknown, it is possible that the inhibitor disrupts the mature telomerase holoenzyme structure, rendering it inactive (data not shown). Interestingly, another recent study demonstrated the presence of telomerase RNA at wnt promoters in a genome-wide RNA based chromatin immunoprecipitation⁷⁹. Studies by Mukherjee et al.⁸ demonstrate that most of the extra-telomeric roles of telomerase require it to be in its native holoenzyme form irrespective, of its role in telomere extension. A catalytically competent hTERT mutant that lacks the nuclear localisation signal was unable to enhance cell proliferation, indicating that nuclear localisation (a requirement for maturation of telomerase) is a prerequisite for functionality⁸. From the inventors' data, it can be seen that there is a direct physical association of telomerase with NFκB complexes (as shown by supershift assays) (FIGS. 6D and E) on endogenous promoters. Hence, the mechanism of telomerase mediated regulation of wnt dependent transcription relies on recruitment of chromatin remodelling factors while its role in NFκB dependent transcription depends on its ability to directly control the strength of NFκB binding to a select group of its promoters. Analysis of stimulation dependent p65 binding on a genome-wide scale in the presence of telomerase inhibition demonstrated significant reduction in p65 binding to a limited subset of target gene promoters (FIG. 5). Interestingly, the ChIP-seq data as well as candidate ChIP data from human cells reveals that telomerase mediated regulation is specific to a subset of NFκB dependent promoters like those of IL6 and TNF. This observation assumes importance since IL6 is a cytokine that has been analysed in depth for its role in tumorigenesis and maintenance of chemo and radio resistant niches involved in metastasis⁸⁰. The inventors explored functional relevance of these findings by analysing IL6 expression in response to chemical inhibition of telomerase in primary haematological malignancies. Indeed, chemical inhibition of telomerase led to attenuation of IL6 in AML, ALL and CML patient samples (FIG. 6F). While inhibition of either telomerase or NFκB is considered an attractive therapeutic strategy in these malignancies, the inventors' results would suggest that telomerase inhibitors could work well due in part to their ability to also inhibit NFκB target genes like IL6.

Inflammation is the latest addition to the list of key changes a cell must acquire to be transformed according to the Hanahan and Weinberg model⁸¹. NFκB is a key transcription factor that orchestrates the inflammatory program in many cells types and particularly in tumor infiltrating macrophages, which aid in tumor cell growth and transformation⁸²⁻⁸⁵. Activation of NFκB, both in tumor cells and in infiltrating immune cells provides a feed forward loop that drives the expression of a number of cancer-related functions. The inventors' observations are summarized in a schematic model as shown in FIG. 6H. Under homeostasis, somatic cells have very little telomerase. In this state, the strength of NFκB dependent gene transcription is sufficient to drive gene expression, which regulates proliferation, resistance to apoptosis and innate immune responses. However, in cancer cells, which require sustained and enhanced activity of NFκB target genes, the reactivated telomerase (which itself is a NFκB target gene) and NFκB form a feed forward loop where telomerase associates with p65 on a subset of target gene promoters (FIG. 6H). This greatly enhances NFκB dependent genes expression and drives cellular proliferation, resistance apoptosis and creates a chronic inflammatory state. This on one hand, in an autocrine manner, activates more NFκB signalling (and hTERT expression) in the cancer cells but on the other hand also causes infiltration of other immune cells like macrophages and hence sets up a state highly favorable to tumor growth and survival. In summary, the inventors' results demonstrate a previously unanticipated role for telomerase in directly regulating inflammation. They provide a unifying explanation for the requirement to reactivate telomerase and sustain inflammation in human cancers.

### REFERENCES

1 de Lange, T. Shelterin: the protein complex that shapes and safeguards human telomeres. Genes Dev 19, 2100-2110, doi:10.1101/gad.1346005 (2005).
2 Verdun, R. E. & Karlseder, J. Replication and protection of telomeres. Nature 447, 924-931, doi:nature05976 [pii] 10.1038/nature05976 (2007).
3 Blackburn, E. H. The end of the (DNA) line. Nat Struct Biol 7, 847-850, doi:10.1038/79594 (2000)
4 Blackburn, E. H. & Collins, K. Telomerase: an RNP enzyme synthesizes DNA. Cold Spring Harb Perspect Biol 3, doi:cshperspect.a003558 [pii] 10.1101/cshperspect.a003558 (2011).
5 Gizard, F. et. al. Telomerase activation in atherosclerosis and induction of telomerase reverse transcriptase expression by inflammatory stimuli in macrophages. Arterioscler Thromb Vasc Biol 31, 245-252, doi:10.1161/ATVBAHA.110.219808 (2011).
6 Maida, Y. et al. An RNA-dependent RNA polymerase formed by TERT and the RMRP RNA. Nature 461, 230-235, doi:10.1038/nature08283 (2009).
7 Nitta, E. et al. Telomerase reverse transcriptase protects ATM-deficient hematopoietic stem cells from ROS-induced apoptosis through a telomere-independent mechanism. Blood 117, 4169-4180, doi:10.1182/blood-2010-08-297390 (2011).
8 Mukherjee, S., Firpo, E. J., Wang, Y. & Roberts, J. M. Separation of telomerase functions by reverse genetics. Proc Natl Acad Sci USA, doi:10.1073/pnas.1112414108 (2011).
9 Masutomi, K. et al. The telomerase reverse transcriptase regulates chromatin state and DNA damage responses. Proc Natl Acad Sci USA 102, 8222-8227, doi:10.1073/pnas.0503095102 (2005).
10 Park, J. I. et al. Telomerase modulates Wnt signalling by association with target gene chromatin. Nature 460, 66-72, doi:10.1038/nature08137 (2009)
11 Bradshaw, P. S., Stavropoulos, D. J. & Meyn, M. S. Human telomeric protein TRF2 associates with genomic double-strand breaks as an early response to DNA damage. Nat Genet 37, 193-197, doi:10.1038/ng1506 (2005).
12 Kaminker, P. et al. Higher-order nuclear organization in growth arrest of human mammary epithelial cells: a novel role for telomere-associated protein TIN2. J Cell Sci 118, 1321-1330, doi:10.1242/jcs.01709 (2005).
13 Teo, H. et al. Telomere-independent Rap1 is an IKK adaptor and regulates NF-kappaB-dependent gene expression. Nat Cell Biol 12, 758-767, doi:10.1038/ncb2080 (2010).
14 Martinez, P. et al. Mammalian Rap1 controls telomere function and gene expression through binding to telomeric and extratelomeric sites. Nat Cell Biol 12, 768-780, doi:10.1038/ncb2081 (2010).
15 Martinez, P. & Blasco, M. A. Telomeric and extra-telomeric roles for telomerase and the telomere-binding proteins. Nat Rev Cancer 11, 161-176, doi:10.1038/nrc3025 (2011).
16 Li, C. T. et al. Vorinostat represses telomerase activity via epigenetic regulation of telomerase reverse transcriptase in non-small cell lung cancer cells. J Cell Biochem, doi:10.1002/jcb.23229 (2011).
17 Indran, I., R., Hande, M. P. & Pervaiz, S. hTERT overexpression alleviates intracellular ROS production, improves mitochondrial function, and inhibits ROS-mediated apoptosis in cancer cells. Cancer Res 71, 266-276, doi:10.1158/0008-5472.CAN-10-1588 (2011).
18 Basseres, D. S., Ebbs, A., Levantini, E. & Baldwin, A. S. Requirement of the NF-kappaB subunit p65/RelA for K-Ras-induced lung tumorigenesis. Cancer Res 70, 3537-3546, doi:0008-5472.CAN-09-4290 [pii] 10.1158/0008-5472.CAN-09-4290 (2010).
19 Begemann, S., Galimi, F. & Karlseder, J. Moderate expression of TRF2 in the hematopoietic system increases development of large cell blastic T-cell lymphomas. Aging (Albany N.Y.) 1, 122-130 (2009).
20 Blackburn, E. H. et al. Recognition and elongation of telomeres by telomerase. Genome 31, 553-560 (1989).
21 Hayflick, L. The Limited in Vitro Lifetime of Human Diploid Cell Strains. Exp Cell Res 37, 614-636 (1965).
22 Harley, C. B. Telomerase and cancer therapeutics. Nat Rev Cancer 8, 167-179, doi:10.1038/nrc2275 (2008).
23 Mitchell, J. R., Wood, E. & Collins, K. A telomerase component is defective in the human disease dyskeratosis congenita. Nature 402, 551-555, doi:10.1038/990141 (1999).
24 Shkreli, M. et al. Reversible cell-cycle entry in adult kidney podocytes through regulated control of telomerase and Wnt signaling. Nat Med 18, 111-119, doi:nm.2550 [pii] 10.1038/nm.2550 (2011).
25 Shay, J. W. & Wright, W. E. Senescence and immortalization: role of telomeres and telomerase. Carcinogenesis 26, 867-874, doi:10.1093/carcin/bgh296 (2005).
26 Counter, C. M., Hirte, H. W., Bacchetti, S. & Harley, C. B. Telomerase activity in human ovarian carcinoma. Proc Natl Acad Sci USA 91, 2900-2904 (1994).
27 Blackburn, E. H., Greider, C. W. & Szostak, J. W. Telomeres and telomerase: the path from maize, Tetrahymena and yeast to human cancer and aging. Nat Med 12, 1133-1138, doi:10.1038/nm1006-1133 (2006).
28 Stewart, S. A. et al. Telomerase contributes to tumorigenesis by a telomere length-independent mechanism. Proc Natl Acad Sci USA 99, 12606-12611, doi:10.1073/pnas.182407599 182407599 [pii] (2002).
29 Chang, S. & DePinho, R. A. Telomerase extracurricular activities. Proc Natl Acad Sci USA 99, 12520-12522, doi:10.1073/pnas.212514699 212514699 [pii] (2002).
30 Gordon, D. M. & Santos, J. H. The emerging role of telomerase reverse transcriptase in mitochondrial DNA metabolism. J Nucleic Acids 2010, doi:10.4061/2010/390791 (2010).
31 Beliveau, A. & Yaswen, P. Soothing the watchman: telomerase reduces the p53-dependent cellular stress response. Cell Cycle 6, 1284-1287 (2007)
32 Chenette, E. J. DNA damage response: Keeping telomerase at bay. Nat Rev Mol Cell Biol 10, 813, doi:10.1038/nrm2801 (2009).
33 Tamakawa, R. A., Fleisig, H. B. & Wong, J. M. Telomerase inhibition potentiates the effects of genotoxic agents in breast and colorectal cancer cells in a cell cycle-specific manner. Cancer Res 70, 8684-8694, doi:10.1158/0008-5472.CAN-10-2227 (2010).
34 Gonzalez-Suarez, E. et al. Increased epidermal tumors and increased skin wound healing in transgenic mice overexpressing the catalytic subunit of telomerase, mTERT, in basal keratinocytes. Embo J 20, 2619-2630doi:10.1093/emboj/20.11.2619 (2001).
35 Artandi, S. E. et al. Constitutive telomerase expression promotes mammary carcinomas in aging mice. Proc Natl Acad Sci USA 99, 8191-8196, doi:10.1073/pnas.112515399 (2002).
36 Gonzalez-Suarez, E., Geserick, C., Flores, J. M. & Blasco, M. A. Antagonistic effects of telomerase on cancer and aging in K5-mTert transgenic mice. Oncogene 24, 2256-2270, doi:1208413 [pii] 10.1038/sj.onc.1208413 (2005).
37 Blasco, M. A., Rizen, M., Greider, C. W. & Hanahan, D. Differential regulation of telomerase activity and telomerase RNA during multi-stage tumorigenesis. Nat Genet 12, 200-204, doi:10.1038/ng0296-200 (1996).
38 Cosme-Blanco, W. et al. Telomere dysfunction suppresses spontaneous tumorigenesis in vivo by initiating p53-dependent cellular senescence. EMBO Rep 8, 497-503, doi:7400937 [pii] 10.1038/sj.embor.7400937 (2007).
39 Herrera, E., Martinez, A. C. & Blasco, M. A. Impaired germinal center reaction in mice with short telomeres. Embo J 19, 472-481, doi:10.1093/emboj/19.3.472 (2000).
40 Goytisolo, F. A. et al. Short telomeres result in organismal hypersensitivity to ionizing radiation in mammals. J Exp Med 192, 1625-1636 (2000).
41 Wynford-Thomas, D. & Kipling, D. Telomerase. Cancer and the knockout mouse. Nature 389, 551-552, doi:10.1038/39207 (1997).
42 Blasco, M. A. et al. Telomere shortening and tumor formation by mouse cells lacking telomerase RNA. Cell 91, 25-34 (1997).
43 Greenberg, R. A., Allsopp, R. C., Chin, L., Morin, G. B. & DePinho, R. A. Expression of mouse telomerase reverse transcriptase during development, differentiation and proliferation. Oncogene 16, 1723-1730, doi:10.103.8/sj.onc.1201933 (1998).
44 Smith, L. L., Coller, H. A. & Roberts, J. M. Telomerase modulates expression of growth-controlling genes and enhances cell proliferation. Nat Cell Biol 5, 474-479, doi:10.1038/ncb985 (2003).
45 Sarin, K. Y. et al. Conditional telomerase induction causes proliferation of hair follicle stem cells. Nature 436, 1048-1052, doi:10.1038/nature03836 (2005).
46 Bodnar, A. G. et al. Extension of life-span by introduction of telomerase into normal human cells. Science 279, 349-352 (1998)
47 Ren, J. G. et al. Expression of telomerase inhibits hydroxyl radical-induced apoptosis in normal telomerase negative human lung fibroblasts. FEBS Lett 488, 133-138 (2001).
48 Roth, A., Harley, C. B. & Baerlocher, G. M. Imetelstat (GRN163L)-telomerase-based cancer therapy. Recent Results Cancer Res 184, 221-234, doi:10.1007/978-3-642-01222-8_{—}16 (2010).
49 Joseph, I. et al. The telomerase inhibitor imetelstat depletes cancer stem cells in breast and pancreatic cancer cell lines. Cancer Res 70, 9494-9504, doi:10.1158/0008-5472.CAN-10-0233 (2010).
50 Esakova, O. & Krasilnikov, A. S. Of proteins and RNA: the RNase P/MRP family. Rna 16, 1725-1747, doi:10.1261/rna.2214510 (2010).
51 Baldwin, A. S., Jr. Series introduction: the transcription factor NF-kappaB and human disease. J Clin Invest 107, 3-6, doi:10.1172/JCI11891 (2001)
52 Perkins, N. D. The diverse and complex roles of NF-kappaB subunits in cancer. Nat Rev Cancer 12, 121-132, doi:nrc3204 [pii] 10.1038/nrc3204 (2012).
53 Oeckinghaus, A., Hayden, M. S. & Ghosh, S. Crosstalk in NF-kappaB signaling pathways. Nat Immunol 12, 695-708, doi:ni.2065 [pii] 10.1038/ni.2065 (2011).
54 McCool, K. W. & Miyamoto, S. DNA damage-dependent NF-kappaB activation: NEMO turns nuclear signaling inside out. Immunol Rev 246, 311-326, doi:10.1111/j.1600-065X.2012.01101.x (2012).
55 Smale, S. T. Dimer-specific regulatory mechanisms within the NF-kappaB family of transcription factors. Immunol Rev 246, 193-204, doi:10.1111/j.1600-065X.2011.01091.x (2012).
56 Barkett, M. & Gilmore, T. D. Control of apoptosis by Rel/NF-kappaB transcription factors. Oncogene 18, 6910-6924, doi:10.1038/sj.onc.1203238 (1999).
57 Hayden, M. S. & Ghosh, S. Shared principles in NF-kappaB signaling. Cell 132, 344-362, doi:S0092-8674(08)00120-7 [pii] 10.1016/j.cell.2008.01.020 (2008).
58 Loercher, A. et al. Nuclear factor-kappaB is an important modulator of the altered gene expression profile and malignant phenotype in squamous cell carcinoma. Cancer Res 64, 6511-6523, doi:10.1158/0008-5472.CAN-04-0852 64/18/6511 [pii] (2004).
59 Jones, R. L. et al. Nuclear NF-kappaB/p65 expression and response to neoadjuvant chemotherapy in breast cancer. J Clin Pathol 64, 130-135, doi:10.1136/jcp.2010.082966 (2011).
60 Ben-Neriah, Y. & Karin, M. Inflammation meets cancer, with NF-kappaB as the matchmaker. Nat Immunol 12, 715-723, doi:10.1038/ni.2060 (2011).
61 Karin, M. NF-kappaB as a critical link between inflammation and cancer. Cold Spring Harb Perspect Biol 1, a000141, doi:10.1101/cshperspect.a0.00141 (2009).
62 Orlowski, R. Z. & Baldwin, A. S., Jr. NF-kappaB as a therapeutic target in cancer. Trends Mol Med 8, 385-389, doi:S1471491402023754 [pii] (2002).
63 Wu, Z. H. et al. ATM- and NEMO-dependent ELKS ubiquitination coordinates TAK1-mediated IKK activation in response to genotoxic stress. Mol Cell 40, 75-86, doi:S1097-2765(10)00712-4 [pii] 10.1016/j.molcel.2010.09.010 (2010).
64 Vanden Berghe, W., De Bosscher, K., Boone, E., Plaisance, S. & Haegeman, G. The nuclear factor-kappaB engages CBP/p300 and histone acetyltransferase activity for transcriptional activation of the interleukin-6 gene promoter. J Biol Chem 274, 32091-32098 (1999).
65 Saccani, S., Marazzi, I., Beg, A. A. & Natoli, G. Degradation of promoter-bound p65/RelA is essential for the prompt termination of the nuclear factor kappaB response. J Exp Med 200, 107-113, doi:10.1084/jem.20040196 jem.20040196 [pii] (2004).
66 Van Antwerp, D. J., Martin, S. J., Kafri, T., Green, D. R. & Verma, I. M. Suppression of TNF-alpha-induced apoptosis by NF-kappaB. Science 274, 787-789 (1996).
67 Seimiya, H. et al. Telomere shortening and growth inhibition of human cancer cells by novel synthetic telomerase inhibitors MST-312, MST-295, and MST-1991. Mol Cancer Ther 1, 657-665 (2002).
68 Pfeffer, K. et al. Mice deficient for the 55 kd tumor necrosis factor receptor are resistant to endotoxic shock, yet succumb to L. monocytogenes infection. Cell 73, 457-467, doi:0092-8674(93)90134-C [pii] (1993).
69 Dejager, L. & Libert, C. Tumor necrosis factor alpha mediates the lethal hepatotoxic effects of poly(I:C) in D-galactosamine-sensitized mice. Cytokine 42, 55-61, doi:S1043-4666(08)00032-X [pii] 10.1016/j.cyto.2008.01.014 (2008).
70 Kong, S. L., Li, G., Loh, S. L., Sung, W. K. & Liu, E. T. Cellular reprogramming by the conjoint action of ERalpha, FOXA1, and GATA3 to a ligand-inducible growth state. Mol Syst Biol 7, 526, doi:msb201159 [pii] 10.1038/msb.2011.59 (2011).
71 Yin, L., Hubbard, A. K. & Giardina, C. NF-kappa B regulates transcription of the mouse telomerase catalytic subunit. J Biol Chem 275, 36671-36675, doi:10.1074/jbc.M007378200 M007378200 [pii] (2000).
72 Terrin, L. et al. Latent membrane protein 1 of Epstein-Barr virus activates the hTERT promoter and enhances telomerase activity in B lymphocytes. J Virol 82, 10175-10187, doi:JVI.00321-08 [pii] 10.1128/JVI.00321-08 (2008).
73 Sinha-Datta, U. et al. Transcriptional activation of hTERT through the NF-kappaB pathway in HTLV-I-transformed cells. Blood 104, 2523-2531, doi:10.1182/blood-2003-12-4251 2003-12-4251 [pii] (2004).
74 Rentoukas, E. et al. Connection between Telomerase Activity in PBMC and Markers of Inflammation and Endothelial Dysfunction in Patients with Metabolic Syndrome. PLoS One 7, e35739, doi:10.1371/journal.pone.0035739 PONE-D-11-25792 [pii] (2012).
75 Ping, L., Asai, A., Okada, A., Isobe, K. & Nakajima, H. Dramatic increase of telomerase activity during dendritic cell differentiation and maturation. J Leukoc Biol 74, 270-276 (2003).
76 Bodnar, A. G., Kim, N. W., Effros, R. B. & Chiu, C. P. Mechanism of telomerase induction during T cell activation. Exp Cell Res 228, 58-64, doi:S0014-4827(96)90299-2 [pii] 10.1006/excr.1996.0299 (1996).
77 Eaton, K. A., Opp, J. S., Gray, B. M., Bergin, I. L. & Young, V. B. Ulcerative typhlocolitis associated with Helicobacter mastomyrinus in telomerase deficient mice. Vet Pathol 48, 713-725, doi:0300985810383876 [pii] 10.1177/0300985810383876 (2011).
78 Beg, A. A., Sha, W. C., Bronson, R. T., Ghosh, S. & Baltimore, D. Embryonic lethality and liver degeneration in mice lacking the RelA component of NF-kappa B. Nature 376, 167-170, doi:10.1038/376167a0 (1995).
79 Chu, C., Qu, K., Zhong, F. L., Artandi, S. E. & Chang, H. Y. Genomic maps of long noncoding RNA occupancy reveal principles of RNA-chromatin interactions. Mol Cell 44, 667-678, doi:S1097-2765(11)00680-0 [pii] 10.1016/j.molcel.2011.08.027 (2011).
80 Gilbert, L. A. & Hemann, M. T. DNA damage-mediated induction of a chemoresistant niche. Cell 143, 355-366, doi:10.1016/j.cell.2010.09.043 (2010).
81 Hanahan, D. & Weinberg, R. A. Hallmarks of cancer: the next generation. Cell 144, 646-674, doi:S0092-8674(11)00127-9 [pii] 10.1016/j.cell.2011.02.013 (2011).
82 Kim, D. W. et al. Activation of NF-kappaB/Rel occurs early during neoplastic transformation of mammary cells. Carcinogenesis 21, 871-879 (2000).
83 Pikarsky, E. et al. NF-kappaB functions as a tumour promoter in inflammation-associated cancer. Nature 431, 461-466, doi:10.1038/nature02924 nature02924 [pii] (2004).
84 DeNardo, D. G., Johansson, M. & Coussens, L. M. Inflaming gastrointestinal oncogenic programming. Cancer Cell 14, 7-9, doi:S1535-6108(08)00197-9 [pii] 10.1016/j.ccr.2008.06.010 (2008).
85 Mantovani, A., Allavena, P., Sica, A. & Balkwill, F. Cancer-related inflammation. Nature 454, 436-444, doi:nature07205 [pii] 10.1038/nature07205 (2008).
86 Li, S., Crothers, J., Haqq, C. M. & Blackburn, E. H. Cellular and gene expression responses involved in the rapid growth inhibition of human cancer cells by RNA interference-mediated depletion of telomerase RNA. J Biol Chem 280, 23709-23717, doi:10.1074/jbc.M502782200 (2005).
87 Li, S. et al. Rapid inhibition of cancer cell growth induced by lentiviral delivery and expression of mutant-template telomerase RNA and anti-telomerase short-interfering RNA. Cancer Res 64, 4833-4840, doi:10.1158/0008-5472.CAN-04-0953 (2004).
88 Tergaonkar, V., Correa, R. G., Ikawa, M. & Verma, I. M. Distinct roles of IkappaB proteins in regulating constitutive NF-kappaB activity. Nat Cell Biol 7, 921-923, doi:ncb1296 [pii] 10.1038/ncb1296 (2005).
89 Fujita, P. A. et al. The UCSC Genome Browser database: update 2011. Nucleic Acids Res 39, D876-882, doi:gkq963 [pii] 10.1093/nar/gkq963 (2011).
90 Xu, H. et al. A signal-noise model for significance analysis of ChIP-seq with negative control. Bioinformatics. 26, 1199-1204, doi:btq128 [pii] 10.1093/bioinformatics/btq128 (2010).
91 Kent, W. J. et al. The human genome browser at UCSC. Genome Res 12, 996-1006, doi:10.1101/gr.229102. Article published online before print in May 2002 (2002).
92 Zhang, Z., Chang, C. W., Goh, W. L., Sung, W. K. & Cheung, E. CENTDIST: discovery of co-associated factors by motif distribution. Nucleic Acids Res 39, W391-399, doi:gkr387 [pii] 10.1093/nar/gkr387 (2011)
93 Simonet, T. et al. The human TTAGGG repeat factors 1 and 2 bind to a subset of interstitial telomeric sequences and satellite repeats. Cell Res 21, 1028-1038, doi:10.1038/cr.2011.40 (2011).
94 McLean, C. Y. et al. GREAT improves functional interpretation of cis-regulatory regions. Nat Biotechnol 28, 495-501, doi:nbt.1630 [pii] 10.1038/nbt.1630 (2010).
95 Lieber and Strauss (1995) Mol. Cell. Biol. 15, 540-551.

1. A method of treating an inflammatory disease and/or cancer in a patient in need thereof, the method comprising administering a telomerase inhibitor to said patient.

2. A method of sensitizing a patient to treatment with an anti-inflammatory drug and/or an anti-cancer drug, the method comprising administering a telomerase inhibitor to said patient.

3. A method of preventing recurrence of an inflammatory disease and/or cancer in a patient in need thereof, the method comprising administering a telomerase inhibitor to said patient.

4. The method according to claim 1, wherein the telomerase inhibitor is selected from the group consisting of: interfering nucleic acid agent, an antibody, a small inorganic molecule, and a peptide nucleic acid (PNA).

5. The method according to claim 4, wherein the interfering nucleic acid agent is selected from the group consisting of a double stranded RNA (dsRNA), an antisense RNA, and a ribozyme.

6. The method according to claim 5, wherein the dsRNA is selected from the group consisting of short hairpin RNA (shRNA), small interfering (siRNA), and micro RNA (miRNA).

7. The method according to claim 6, wherein the shRNA is selected from the group consisting of SEQ ID NO: 48 and SEQ ID NO: 49.

8. The method according to claim 6, wherein the siRNA is directed against hTERT.

9. The method according to claim 8, wherein the siRNA is selected from the group consisting of SEQ ID NO: 50, SEQ ID NO: 51, SEQ ID NO: 52, and SEQ ID NO: 53.

10. The method according to claim 4, wherein the interfering nucleic acid agent is a 2′-O-alkyl oligonucleotide inhibitor.

11. The method according to claim 4, wherein the small inorganic molecule inhibitor is selected from the group consisting of N,N′-1,3-Phenylenebis-[2,3-dihydroxy-benzamide] (MST-312), BIBR 1532, (2-[(E)-3-naphtalen-2-yl-but-2-enoylamino]-benzoic acid), and costunolide ((3aS,6E,10E,11aR)-6,10-dimethyl-3-methylene-3,3a,4,5,8,9-hexahydrocyclodeca[b]furan-2(11aH)-one).

12. The method according to claim 4, wherein the telomerase inhibitor is based on a compound selected from the group consisting of:

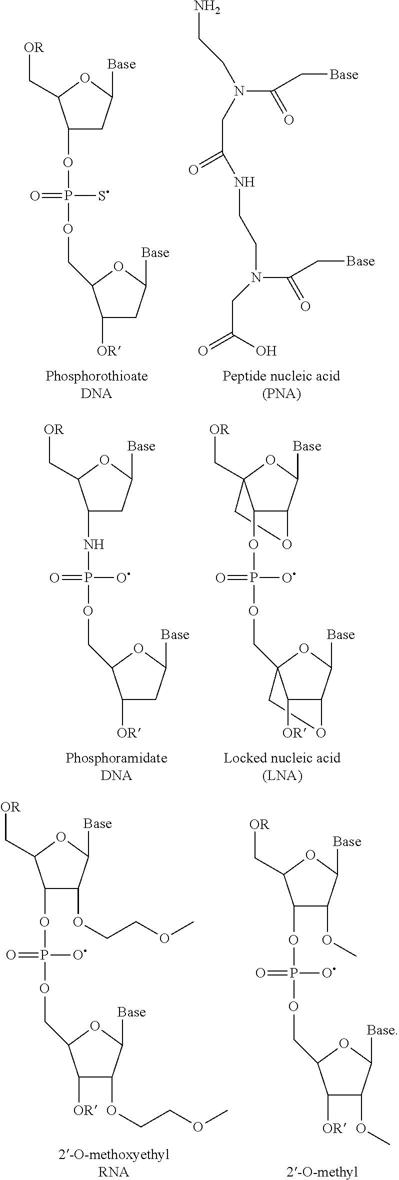

13. The method according to claim 1, comprising administering the telomerase inhibitor with an inhibitor of NFκB.

14. The method according to claim 13, wherein the NFκB inhibitor is selected from the group consisting of p65 shRNA (sc-29410-SH), sc-3060 (sequence: AAVALLPAVLLALLAPVQRKRQKLMP, SEQ ID NO: 47), 2-(1,8-naphthyridin-2-yl)-Phenol, 5-Aminosalicylic acid, BAY 11-7082, BAY 11-7085, CAPE (Caffeic Acid Phenethylester), Diethylmaleate, IMD 0354, Lactacystin, MG-132 [Z-Leu-Leu-Leu-CHO], parthenolide, phenylarsine oxide, PPM-18, Pyrrolidinedithiocarbamic acid ammonium salt, (E)-3-(4-methylphenylsulfonyl)-2-propenenitrile, tetrahydrocurcuminoids, sulfasalazine, sulindac, clonidine, helenalin, wedelolactone, pyrollidinedithiocarbamate (PDTC), Calbiochem IKK-2 inhibitor VI, and Calbiochem IKK inhibitor III (BMS-345541).

15. The method according to claim 1, wherein the inflammatory disease is selected from the group consisting of: an inflammatory disease of the joints, an inflammatory disease of the skin, an inflammatory disease of the eyes, an inflammatory disease of the peripheral or central nervous system, an inflammatory disease of the airways or the lung, and an inflammatory disease of the gastrointestinal tract.

16. The method according to claim 1, wherein the cancer is selected from the group consisting of: acute and chronic leukaemia, bone tumor, breast cancer, colon cancer, lung cancer, prostate cancer, and stomach cancer.

17-19. (canceled)
